(19) United States
(12) Patent Application Publication
(10) Pub. No.: US 2012/0077863 A1
(43) Pub. Date: Mar. 29, 2012

(54) GUANIDINO-SUBSTITUTED BI-AND POLYPHENYLS AS SMALL MOLECULE CARRIERS

(75) Inventors: David Selwood, London (GB); Roberta Worthington, London (GB); Anne-Sophie Rebstock, London (GB); Michela Simone, London (GB); Matt Gooding, London (GB); Henry Dube, London (GB); Kai Stoeber, London (GB); Slavica Tudzarova, London (GB); Christoffer Boshoff, London (GB); Gareth Williams, London (GB); Sarah Kingsbury, London (GB); Dimitrios Lagos, London (GB); Juan Manuel Funes Quesada, London (GB)

(73) Assignee: UCL Business PLC, London (GB)

(21) Appl. No.: 13/203,667

(22) PCT Filed: Mar. 1, 2010
(86) PCT No.: PCT/GB2010/000359

§ 371 Date: Dec. 7, 2011

(30) Foreign Application Priority Data
Feb. 27, 2009 (GB) 0903482.8

(51) Int. Cl. A61K 31/713; C12N 5/071; A61K 31/155; C07C 279/08; C07H 21/02

(52) U.S. Cl. 514/44 A; 564/236; 536/24.5; 514/634; 435/375

(57) ABSTRACT

A compound of formula I, or a pharmaceutically acceptable salt thereof, wherein —X₁, X₂ and X₃ are each independently where Y is an alkylene, alkenylene or alkynylene group, each of which may be optionally substituted with one or more substituents selected from alkyl, halo, CF₃, OH, alkoxy, NH₂, CN, NO₂ and COOH; W is absent or is O, S or NH; R₁, R₂, R₃ and R₄ are each independently selected from H, alkyl, aryl and a protecting group Pi; R₇, R₈ and R₉ are each independently selected from H, alkyl, halo, CF₃, OH, alkoxy, CN, NO₂ and COOH; —q is 1, 2, 3 or 4; q′ is 0, 1, 2 or 3, where q+q′ equals 4; p and r are each independently 1, 2, 3, 4 or 5; p′ and r′ are each independently 0, 1, 2, 3 or 4, where p+p′ and r+r′ each equal 5; and —n is 0, 1, 2, 3, 4, 5 or 6.

### BACKGROUND

The present invention relates to small molecule carrier (SMOC) compounds. More specifically, the invention relates to SMOCs that are useful for the in vitro and in vivo delivery of various cargo moieties into cells.

Over recent years, studies have shown that a variety of peptides, many of which are present in viral proteins, have the ability to cross biological membranes in various different cell types. These peptides, known as “protein transduction domains” (PTDs), can be linked to a wide variety of molecules with limited ability to cross membranes, (e.g., peptides, proteins, DNA), thereby enabling them to traverse biological membranes. Studies have shown that PTD fusion molecules introduced into mice exhibit delivery to all tissues, including the traversal of the blood-brain barrier [Schwarze, S R., Dowdy, S F., Trends Pharmacol. Sci, 2000, 21, 45]. Similar basic peptides are known to have anti-bacterial activity against MDR forms.

Most therapeutic drugs are limited to a relatively narrow range of physical properties. By way of example, they must be sufficiently polar for administration and distribution, but sufficiently non-polar so as to allow passive diffusion through the relatively non-polar bilayer of the cell. As a consequence, many promising drug candidates (including many peptide drugs) fail to advance clinically because they fall outside of this range, proving to be either too non-polar for administration and distribution, or too polar for passive cellular entry. An approach to circumvent this problem is to covalently tether these potential drugs to PTDs. However, it is very costly and time consuming to prepare such peptide-PTDs and their peptide structure often renders them susceptible to rapid degradation by cellular enzymes.

One solution to this problem is to use small molecule carriers (SMOCs or “molecular tugs”) that are more amenable than peptide-PTDs due to their in vivo stability by virtue of their resistance to cellular enzymes that degrade peptides.

WO-A-05123676, which is incorporated herein by reference, describes SMOC compounds and a process for their production.

Rebstock, et al, ChemBioChem 2008, 9(10:1787-1796, which is also incorporated herein by reference, describes an improved synthesis of the SMOC compounds described in WO-A-05123676. Synthetic techniques for preparing the SMOC compounds are also disclosed in the international patent application PCT/GB08/002,911 claiming priority from GB 0716783.6, which is also incorporated herein by reference.

These previous approaches have all involved SMOC compounds physically linked to the cargo they transport with covalent bonds. However, the inventors have now surprisingly found that certain SMOC compounds complex non-covalently to cargo moieties allowing the transport of cargo moieties into cells.

Advantages of these non-covalent conjugates over covalent conjugates include ease of preparation and more rapid release of the cargo moiety inside the cell.

This invention is applicable to a wide range of cargo moieties as detailed herein, and especially to siRNA, which does not cross cell membranes easily, even at high concentrations. The present invention allows the delivery of siRNA to multiple cell types not currently accessible using known approaches.

### SUMMARY OF THE INVENTION

The present invention therefore provides a compound of formula I, or a pharmaceutically acceptable salt thereof,

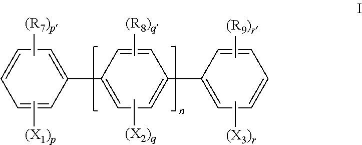

wherein
X₁, X₂ and X₃ are each independently

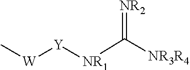

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, CN, NO₂ and COOH;
q is 1, 2, 3 or 4;
q′ is 0, 1, 2 or 3, where q+q′ equals 4;
p and r are each independently 1, 2, 3, 4 or 5;
p′ and r′ are each independently 0, 1, 2, 3 or 4, where p+p′ and r+r′ each equal 5; and
n is 0, 1, 2, 3,4, 5 or 6.

Also provided is a conjugate (U) comprising a compound of formula I as defined above and a cargo moiety selected from a protein, a peptide, an oligonucleotide, a nucleotide, a diagnostic agent and a drug.

### BRIEF DESCRIPTION OF THE FIGURES

FIG. 1 shows the results of a gel electrophoresis binding strength analysis comparing the binding between siRNA targeted to the cdc 25A and cdc 7 genes with (i) the 4G SMOC compound of the invention shown in Example 2, (ii) (Arg)₄ and (iii) (Arg)₈.

FIG. 2 shows the results of an isothermal calorimetry experiment carried out to analyse the strength of the binding between the 6G SMOC compound of the invention shown in Example 3 and siRNA targeted to the cdc25A and cdc7 genes.

FIG. 3 shows the cellular uptake, followed via live microscopy, of a 4G SMOC compound of the invention in a WI-38 human diploid fibroblast cell line.

FIG. 4 shows a comparison of the efficiency of knockdown of cdc7 mRNA using (i) a complex of a SMOC compound of the invention with siRNA targeted to the cdc7 gene and (ii) a complex of lipofectamine with the same siRNA.

FIG. 5 shows a comparison of the protein level (as assessed by Westerns) of (i) a complex of a SMOC compound of the invention with siRNA targeted to the cdc7 gene and (ii) a complex of lipofectamine with the same siRNA.

FIG. 6 shows a comparison of the progression through the cell cycle of (i) a complex of a SMOC compound of the invention with siRNA targeted to the cdc7 gene and (ii) a complex of lipofectamine with the same siRNA.

### DETAILED DESCRIPTION OF THE INVENTION

Typically, in the conjugate (U), the compound of formula I is non-covalently bound to the cargo moiety.

Also provided is a compound of formula A₁-B-A₂, or a pharmaceutically acceptable salt thereof, wherein A₁ and A₂ may be the same or different and are

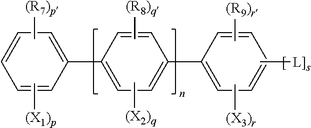

wherein:
R₇, R₈, R₉, X₁, X₂, X₃, n, p, p′, q, q′, r and r′ are as defined above;
S is 0 or 1;
r+r′+s is 5;
L is (Z)ₘNR₅R₆ where Z is a hydrocarbyl group and m is 0 or 1;
   where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore,

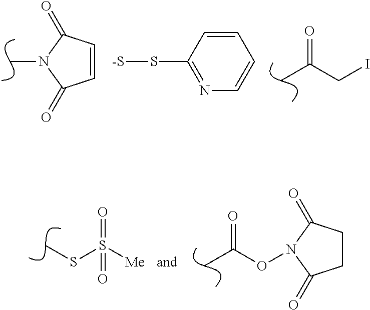

or R₅, R₆ and the nitrogen to which they are attached together form; and

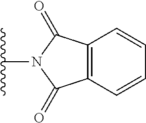

B is —Y′-D-Y′-E-Y′-F-Y′—, wherein:
each Y′ is the same or different and represents a direct bond or a C₁-C₄ alkylene group, which is unsubstituted or substituted with one or more substituents selected from halogen atoms, —NH₂ and —OH groups;
D and F are each independently chosen from:
(i) —NR₁₁C(O)—, —C(O)NR₁₁—, —C(O)O—, —OC(O)—, —O—, —S—, —NR₁₂—, —CO—, —S(O), —S(O)₂—, wherein R₁₁ and R₁₂ each independently represents a hydrogen atom or a straight or branched C₁₋₆ alkyl group, which is unsubstituted or substituted with one or more substituents selected from halogen atoms, —NH₂ and —OH groups; or
(ii) 5- to 6-membered heteroaryl or 5- to 6-membered non-aromatic heterocyclic groups, optionally substituted by one, two or three substituents selected from halogen atoms, hydroxy, —SH, —NH₂, nitro, cyano, straight or branched C₁₋₆ alkyl, straight or branched C₁₋₆ alkoxy, which C₁₋₆ alkyl and C₁₋₆ alkoxy groups may themselves be substituted with one or more substituents selected from halogen atoms, —NH₂ and —OH groups; and
E is chosen from —S—S— and a direct bond.

Also provided is a conjugate (V) comprising a compound of formula A₁-B-A₂ as defined above and a cargo moiety as defined above.

Typically, in the conjugate (V), the compound of formula A₁-B-A₂ is non-covalently bound to the cargo moiety, Typically, in the conjugate (V), s is 0.

Also provided is a process for preparing a conjugate (W), which process comprises reacting a compound of formula A₁-B-A₂ as defined above or a pharmaceutically acceptable salt thereof, in which s is 1 and L is other than a moiety —(Z)ₘ-phthalimide, wherein Z and in are as defined above, with a cargo moiety as defined above.

Also provided is a conjugate (W) obtainable by reacting a compound of formula A₁-B-A₂ as defined above or a pharmaceutically acceptable salt thereof, in which s is 1 and L is other than a moiety —(Z)ₘ-phthalimide, wherein Z and m are as defined above, with a cargo moiety as defined above.

As used herein, the term “hydrocarbyl” refers to a saturated or unsaturated, straight-chain, branched, or cyclic group comprising at least C and H that may optionally comprise one or more other suitable substituents. Examples of such substituents may include halo, alkoxy, hydroxy, CF₃, CN, amino, COOH, nitro or a cyclic group. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon.

Preferably, the hydrocarbyl group is an aryl or alkyl group. Typically, the hydrocarbyl group is unsubstituted. More preferably, the hydrocarbyl group is an unsubstituted C₁₋₆ alkyl group.

As used herein, the term “alkyl” includes both saturated straight chain and branched alkyl groups which may be unsubstituted or substituted (mono- or poly-) by one or more halogen atoms, or CF₃, OH, alkoxy, CN, NO₂, COOH or alkyl substituents. Typically, said alkoxy and alkyl substituents are themselves unsubstituted or substituted with one or more halogen atoms, or OH groups. Preferably, though, said alkoxy and alkyl substituents are themselves unsubstituted.

Preferably, said alkyl groups are unsubstituted or substituted (mono- or poly-) by one or more, preferably 1 or 2, halogen atoms or OH groups.

More preferably, said alkyl groups are unsubstituted.

Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. The term “alkylene” should be construed accordingly.

Most preferably, the alkyl group is an unsubstituted C₁₋₄ alkyl group.

As used herein, an alkoxy group is a said alkyl group, for example a C₁-C₄ or C₁-C₂ alkyl group, which is attached to an oxygen atom.

Preferably, the alkoxy group is a C₁₋₂₀ alkoxy group, more preferably a C₁₋₁₅ alkoxy group, more preferably still a C₁₋₁₂ alkoxy group, more preferably still, a C₁₋₆ alkoxy group, more preferably a C₁₋₃ alkoxy group. Particularly preferred alkoxy groups include, for example, methyoxy, ethyoxy, propoxy, isopropoxy, butoxy, isobutoxy, tert-butoxy, pentoxy and hexoxy. Preferably, the alkoxy group is unsubstituted. More preferably, the alkoxy group is an unsubstituted C₁₋₄ alkoxy group.

As used herein, a haloalkyl group is a said alkyl group, for example a C₁-C₄ or C₁-C₂ alkyl group, which is attached to 1, 2 or 3 halogen atoms.

Preferably, said haloalkyl group is chosen from —CCl₃ and —CF₃.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group. Suitable substituents include alkyl, halo, CF₃, OH, alkoxy, NH₂, CN, NO₂ and COOH. The term “alkenylene” should be construed accordingly. Preferably, the alkenyl group is unsubstituted. More preferably, the alkenyl group is an unsubstituted C₂₋₄ alkenyl group.

As used herein, the term “alkynyl” refers to a carbon chain containing one or more triple bonds, which may be branched or unbranched, and substituted (mono- or poly-) or unsubstituted. Preferably the alkynyl group is a C₂₋₂₀ alkynyl group, more preferably a C₂₋₁₅ alkynyl group, more preferably still a C₂₋₁₂ alkynyl group, or preferably a C₂₋₆ alkynyl group or a C₂₋₃ alkynyl group. Suitable substituents include alkyl, halo, CF₃, OH, alkoxy, NH₂, CN, NO₂ and COOH. The term “alkynylene” should be construed accordingly. Preferably, the alkynyl group is unsubstituted. More preferably, the alkynyl group is an unsubstituted C₂₋₄ alkynyl group.

As used herein, the term “aryl” is a C₆₋₁₀ monoaromatic or polyaromatic system, wherein said polyaromatic system may be fused or unfused, which aryl group may be unsubstituted or substituted by one, two or three substituents selected from halogen atoms, hydroxy, —SH, —NH₂, nitro, cyano, straight or branched C₁₋₆ alkyl, straight or branched C₁₋₆ alkoxy, which C₁₋₆ alkyl and C₁₋₆ alkoxy groups may themselves be substituted with one or more substituents selected from halogen atoms, —NH₂ and —OH groups.

Preferably, said aryl groups are unsubstituted or substituted with one, two or three substituents, which are themselves unsubstituted, selected from halogen atoms, hydroxyl, —NH₂, C₁₋₄ alkyl, C₁₋₄ alkoxy and C₁₋₄ haloalkyl.

More preferably, said aryl groups are unsubstituted or substituted with one or two substituents, which are themselves unsubstituted, selected from halogen atoms, C₁₋₄ alkyl and C₁₋₄ alkoxy.

When an aryl group carries 2 or more substituents, the substituents may be the same or different.

Most preferably, said aryl groups are unsubstituted.

Examples of aryl groups are phenyl, and naphthyl.

In one embodiment, the aryl group is a phenyl group, which is substituted with one or two substituents, which are themselves unsubstituted, selected from halogen atoms, C₁₋₄ alkyl and C₁₋₄ alkoxy.

In another more preferred embodiment, the aryl group is an unsubstituted phenyl group.

As used herein, the term heteroaryl is typically a 5- to 6-membered ring system containing at least one heteroatom selected from O, S and N, which heteroaryl group may be unsubstituted or substituted by one, two or three substituents selected from halogen atoms, hydroxy, —SH, —NH₂, nitro, cyano, straight or branched C₁₋₆ alkyl, straight or branched C₁₋₆ alkoxy, which C₁₋₆ alkyl and C₁₋₆ alkoxy groups may themselves be substituted with one or more substituents selected from halogen atoms, —NH₂ and —OH groups.

Preferably, said heteroaryl groups are unsubstituted or substituted with one, two or three substituents, which are themselves unsubstituted, selected from halogen atoms, hydroxyl, —NH₂, C₁₋₄ alkyl, C₁₋₄ alkoxy and C₁₋₄ haloalkyl.

More preferably, said heteroaryl groups are unsubstituted or substituted with one or two substituents, which are themselves unsubstituted, selected from halogen atoms, C₁₋₄ alkyl and C₁₋₄ alkoxy.

When a heteroaryl group carries 2 or more substituents, the substituents may be the same or different.

Most preferably, said heteroaryl groups are unsubstituted.

Examples of heteroaryl groups are pyridyl, pyrazinyl, pyrimidinyl, pyridazinyl, furyl, oxadiazolyl, oxazolyl, imidazolyl, thiazolyl, thiadiazolyl, thienyl, pyrrolyl, pyridinyl, triazolyl, tetrazolyl, and pyrazolyl groups.

As used herein, the term non-aromatic heterocyclic group is a non-aromatic, saturated or unsaturated C5-C6 carbocyclic ring, in which one or more, for example 1, 2, 3 or 4 of the carbon atoms preferably 1 or 2 of the carbon atoms are replaced by a heteroatom selected from N, O and S, which heterocyclic group may be unsubstituted or substituted by one, two or three substituents selected from halogen atoms, hydroxy, —SH, —NH₂, nitro, cyano, straight or branched C₁₋₆ alkyl, straight or branched C₁₋₆ alkoxy, which C₁₋₆ alkyl and C₁₋₆ alkoxy groups may themselves be substituted with one or more halogen atoms, or —NH₂ or —OH groups. Unsaturated heterocyclyl groups are preferred.

Preferably, said non-aromatic heterocyclic groups are unsubstituted or substituted with one, two or three substituents, which are themselves unsubstituted, selected from halogen atoms, hydroxyl, —NH₂, C₁₋₄ alkyl, C₁₋₄ alkoxy and C₁₋₄ haloalkyl.

More preferably, said non-aromatic heterocyclic groups are unsubstituted or substituted with one or two substituents, which are themselves unsubstituted, selected from halogen atoms, C₁₋₄ alkyl and C₁₋₄ alkoxy.

When a non-aromatic heterocyclic group carries 2 or more substituents, the substituents may be the same or different.

Most preferably, said non-aromatic heterocyclic groups are unsubstituted.

Examples of non-aromatic heterocyclic groups include piperidyl, pyrrolidyl, pyrrolinyl, piperazinyl, morpholinyl, thiomorpholinyl, pyrazolinyl, pirazolidinyl, cromanyl, isocromanyl, imidazolidinyl, 4,5-dihydro-oxazolyl and 3-aza-tetrahydrofuranyl.

As used herein, the term halogen atom embraces chlorine, fluorine, bromine or iodine atoms typically a fluorine, chlorine or bromine atom, most preferably chlorine or fluorine. The term halo when used as a prefix has the same meaning.

As used herein, the term “chromophore” refers to any functional group that absorbs light, giving rise to colour. Typically, the term refers to a group of associated atoms which can exist in at least two states of energy, a ground state of relatively low energy and an excited state to which it may be raised by the absorption of light energy from a specified region of the radiation spectrum. Often, the group of associated atoms contains delocalised electrons. The chromophore present in the compounds prepared by the process of the invention can be a conjugated Π system or a metal complex. Typically, a chromophore is a porphyrin, a polyene, a polyyne or a polyaryl. Preferably the chromophore is one of.

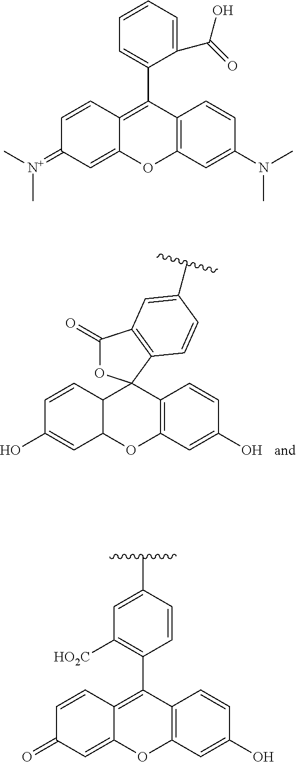

Typically, Y is unsubstituted.

Preferably, Y is a C₁₋₁₀ alkylene group, a C₂₋₁₀ alkenylene group or a C₂₋₁₀ alkynylene group.

More preferably, Y is a C₁₋₁₂ alkylene group, more preferably a C₁₋₁₀ alkylene group, even more preferably a C₁₋₆ alkylene group, and more preferably still, CH₂CH₂.

Preferably, W is O.

Preferably, R₁, R₂, R₃ and R₄ are each independently selected from H and a protecting group P₁. More preferably, R₁ and R₃ are hydrogen and R₂ and R₄ represent H or P₁. Most preferably, R₁ and R₃ are hydrogen and R₂ and R₄ are each independently selected from H and a butyloxycarbonyl (Boc) protecting group.

Preferably, p, q and r are each independently 1 or 2.

In one preferred embodiment, p, q and r are all equal to 1.

In another preferred embodiment, p, q and r are all equal to 2.

In a further preferred embodiment, r is equal to 1 and p is equal to 2.

Preferably, R₇, R₈ and R₉ are each independently selected from H, unsubstituted C₁-C₆ alkyl, unsubstituted C₁-C₆ alkoxy, —CF₃, —CN, halo and OH, more preferably H, —CN or unsubstituted C₁-C₆ alkyl, even more preferably H or —CN, most preferably H.

In one embodiment, R₇ is —CN and R₈ and R₉ are each independently selected from H, alkyl, halo, CF₃, OH, alkoxy, CN, NO₂ and COOH. Preferably, R₇ is —CN and R₈ and R₉ are H.

Preferably, X₁, X₂ and X₃ are the same and are all

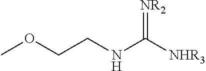

where R₂ and R₃ are each independently H or a Boc protecting group.

Preferably, n is 0, 1, 2 or 3 more preferably 0, 1 or 2, most preferably 1 or 2.

In one particularly preferred embodiment, n is 0.

In another particularly preferred embodiment, n is 1.

In yet another particularly preferred embodiment, n is 2.

Preferably, the compound of formula I is of formula Ia, Ib, Ic, Id, or Ie,

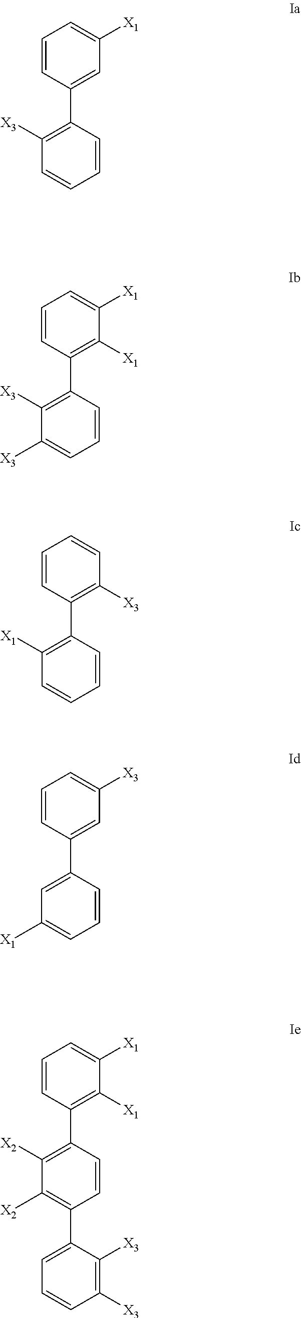

wherein X₁ X₂ and X₃ are as defined above. Compounds of formulae Ib and Ie are preferred.

More preferably, X₁, X₂ and X₃ are the same and are both,

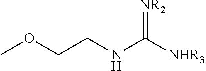

where R₂ and R₃ are each independently H or a Boc protecting group.

In a more preferred embodiment, n is 0 and p+r equals 3. Typically, p=1 and r=2, or p=2 and r=1.

Especially preferred are compounds of formula I selected from the following:

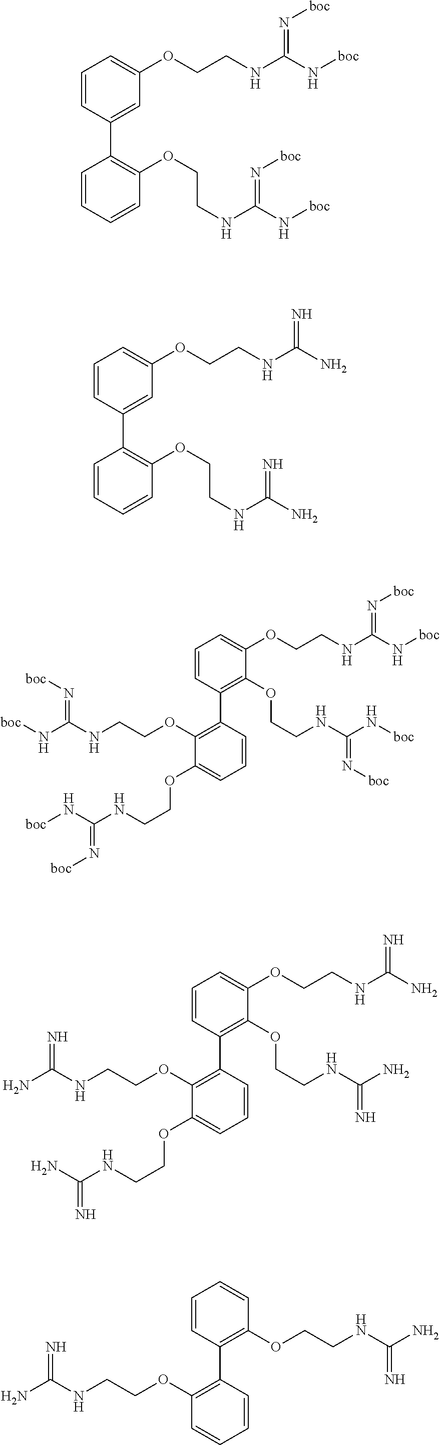

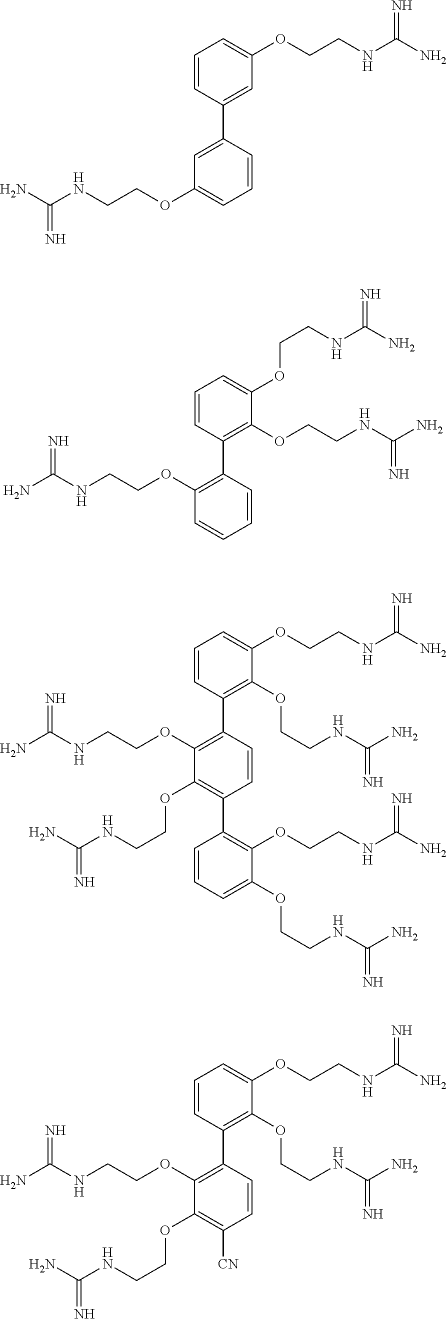

Most preferably, the compound of formula I is selected from:

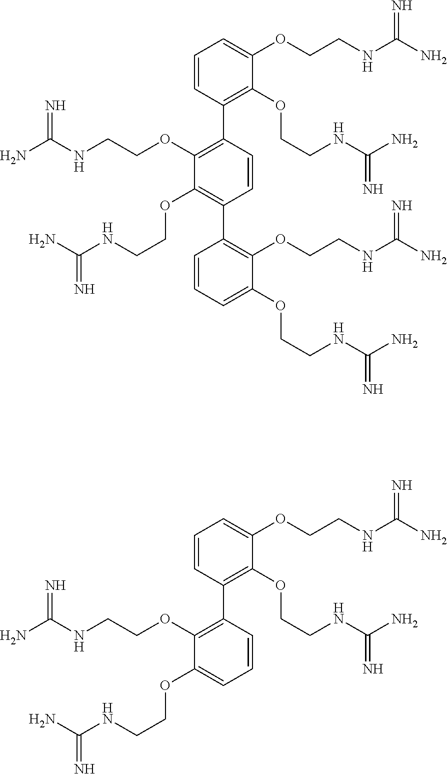

Compounds of formula I may be prepared by known methods, for example by analogous processes to those described in Rebstock, et al, ChemBioChem 2008, 9(11):1787-1796 or WO-A-05123676. Analogous synthetic techniques for preparing SMOC compounds are also disclosed in the international patent application PCT/GB08/002911 claiming priority from GB 0716783.6.

Compounds of formula I are typically prepared by:
(a) coupling a compound of formula II to a compound of formula III to form a compound of formula IV

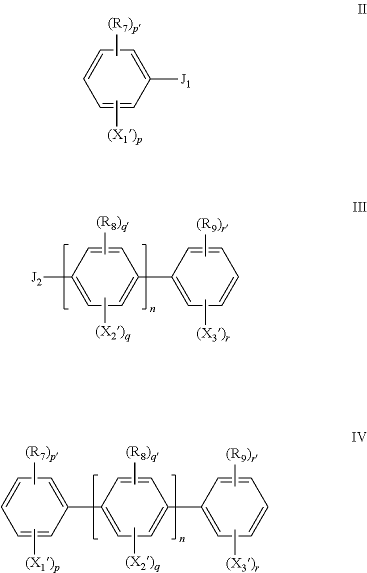

wherein:
n, p, p′, q, q′, r and r′ are defined as above;
at least one X₁′, X₂′ and X₃′ moiety is —W—Y—NR₁R₁₀ or —W—Y—NR₁—C(═NR₂)—NR₃R₄ and the other X₁′, X₂′ and X₃′ moieties are each independently selected from OH, SH, NH₂, —W—Y—NR₁R₁₀, and —W—Y—NR₁—C(═NR₂)—NR₃R₄ wherein R₁, W and Y are defined as above and R₁₀ is H or a protecting group P₂; and
   one of J₁ and J₂ is a leaving group LG₁, and the other is a boronic acid, a boronic ester, a borane group or a trihalogenoborate salt group;
(b) if any of X₁, X₂′ and X₃′ in the formula (IV) is other than —W—Y—NR₁—C(═NR₂)—NR₃R₄, alkylating the X₁′, X₂′ and X₃′ moieties so that they each represent a group of formula

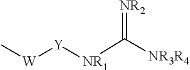

where W, Y, R₁, R₂, R₃ and R₄ are defined as above, to obtain a compound of formula I.

Protecting groups P₁ and P₂ are protecting groups suitable for protecting nitrogen atoms. Many examples of such protecting groups are known to the person skilled in the art, for example those protecting groups mentioned in “Protecting Group Chemistry” Jeremy Robertson, OUP, 2000, which is incorporated herein by reference. Preferably P₁ is selected from benzyl, trityl, 9-phenylfluorenyl, benzydryl, fluorenyl, carbamate, benzylcarbamate (Cbz), t-butyl carbamate (Boc), 9-fluorenylmethyl carbamate (Fmoc), acetamide, p-toluenesulfonate (p-Ts), silyl and triisopropylsilyl (TIPS) groups.

Preferably, P₁ is a Boc group.

Preferably, P₂ is a Cbz group.

Typically P₁ and P₂ are different.

Leaving group LG₁ is typically any group that can undergo oxidative addition with Pd(0). Those of skill in the art will easily be able to select appropriate leaving groups. LG₁ is preferably a halogen, triflate (OTf), tosylate (OTs), N-hydroxysuccinimide (OSu) or a mesylate (OMs) group. LG₁ is more preferably halogen, most preferably bromide or iodide.

Leaving group LG₃ is typically a leaving group suitable for a nucleophilic substitution reaction at a saturated carbon centre. Those of skill in the art will easily be able to select appropriate leaving groups. LG₃ is preferably a halogen, triflate (OTf), tosylate (OTs), N-hydroxysuccinimide (OSu) or a mesylate (OMs) group. LG₃ is more preferably a OMs group.

Leaving group LG₄ can be any leaving group suitable for a guanidinylation reaction. The skilled reader will appreciate that LG₄ represents a moiety such that —NLG₄ is a leaving group in guanidinylation reaction. A skilled chemist can easily select appropriate leaving groups in this regard. Thus, preferred LG₄ groups include triflyl (Tf), tosyl (Ts) and mesyl (Ms) groups. LG₄ is most preferably a triflyl group, such that —NLG₄ represents —NTf.

Leaving group LG₄′ can be any leaving group suitable for a guanidinylation reaction. A skilled chemist can easily select appropriate leaving groups in this regard. LG₄′ is typically a halogen atom, triflate (OTf), tosylate (OTs), mesylate (OMs) or 1-pyrazole group, preferably a 1-pyrazole group.

Leaving group LG₇ is typically a leaving group suitable for a nucleophilic substitution reaction at a saturated carbon centre. Those of skill in the art will easily be able to select appropriate leaving groups. LG₇ is preferably a halogen, triflate (OTf), tosylate (OTs), N-hydroxysuccinimide (OSu) or a mesylate (OMs) group. LG₇ is more preferably a OMs group.

The coupling reaction between the compounds of formulae (II) and (III) is typically a Suzuki reaction performed using a Pd(0) catalyst in the presence of a base.

In one embodiment, when any of the X₁′, X₂′ and X₃′ moieties in the formula (IV) are OH, SH or NH₂, alkylation so that all X₁′, X₂′ and X₃′ moieties represent a group of formula —W—Y—NR₁—C(═NR₂)—NR₃R₄ is effected by either:
(i) alkylating any X₁′, X₂′ and X₃′ moieties that are OH, SH or NH₂ so that they represent —W—Y—NR₁R₁₀, deprotecting any protected amine groups on —W—Y—NR₁R₁₀ moieties present at the X₁′, X₂′ and X₃′ positions and guanidinylating the deprotected moieties so that they each represent a group of formula —W—Y—NR₁—C(═NR₂)—N NR₃R₄; or
(ii) alkylating any X₁′, X₂′ and X₃′ moieties that are OH, SH or NH₂ so that they each represent a group of formula —W—Y—NR₁—C(═NR₂)—NR₃R₄, deprotecting any protected amine groups on —W—Y—NR₁R₁₀ moieties present at the X₁′, X₂′ and X₃′ positions and guanidinylating the deprotected moieties so that they each represent a group of formula —W—Y—NR₁—C(═NR₂)—NR₃R₄.

Typically, the alkylation of hydroxy, thiol and amino groups at the X₁′, X₂′ and X₃′ positions in the formula IV so that they represent —W—Y—NR₁R₁₀ is effected with a compound of formula LG₃-Y—NR₁R₁₀ where R₁, Y and LG₃ are defined as above and R₁₀ is H or a protecting group P₂.

Preferably, the alkylation of any X₁′, X₂ and X₃′ moieties that are OH, SH or NH₂ so that they each represent a group of formula —W—Y—NR₁—C(═NR₂)—NR₃R₄ is effected by a compound of formula LG₇-Y—NR₁—C(═NR₂)—NR₃R₄ where R₁₋₄, LG₇ and Y are as defined above.

Deprotection of any amine groups in the —W—Y—NR₁R₁₀ moieties present at the X₁′, X₂′ and X₃′ positions can be carried out by standard techniques.

Typically, said guanidinylation of deprotected —W—Y—NR₁R₁₀ moieties at the X₁′, X₂′ and X₃′ positions is effected either by a compound of formula V, or a tautomer thereof, where R₂, R₃ and R₄ are as defined in claim 1 and LG₄ is a leaving group;

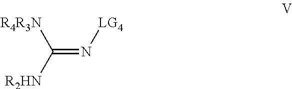

or by a compound of formula V′, or a tautomer thereof,

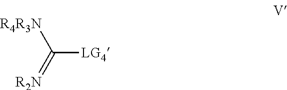

where R₂, R₃ and R₄ are as defined above and LG₄′ is a leaving group.

Preferred tautomers of the compounds of formula (V) are represented by the formula

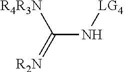

Preferably, guanidinylation is effected with N,N-di-boc-N′-trifluoromethanesulfonyl guanidine or N,N′-Di-Boc-1H-pyrazole-1-carboxamidine, most preferably N,N′-Di-Boc-1H-pyrazole-1-carboxamidine.

The conjugate (U) of the invention typically comprises a compound of formula I as defined above non-covalently bound to a cargo moiety by hydrophobic interactions, pi-pi interactions, cation-pi interactions, hydrogen bonding, ionic interactions, van der Waal's forces or dipole-dipole interactions.

For avoidance of doubt, the compound of formula A₁-B-A₂ is a compound of formula A₁-Y′-D-Y′-E-Y′-F-Y′-A₂.

As the skilled reader will be aware, the formula

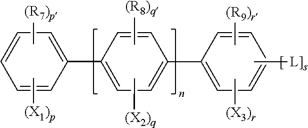

depicted for A₁ and A₂ is attached to the moiety B in the compound A₁-B-A₂. Thus, a hydrogen atom in the formula depicted above will necessarily be replaced by the moiety B.

Typically, in the compound of formula A₁-B-A₂, the moiety B is attached to phenyl rings in A₁ and A₂.

When D is a group —NR₁₁C(O)—, the nitrogen atom is bonded to the Y′ moiety which is, in turn bonded to the A₁ moiety and the carbon atom is bonded to the Y′ moiety which is, in turn bonded to the moiety -E-.

When D is a group —C(O)NR₁₁—, the carbon atom is bonded to the Y′ moiety which is, in turn bonded to the A₁ moiety and the nitrogen is bonded to the Y′ moiety which is, in turn bonded to the moiety -E-.

When D is a group —C(O)O—, the carbon atom is bonded to the Y′ moiety which is, in turn bonded to the A₁ moiety and the oxygen atom is bonded to the Y′ moiety which is, in turn bonded to the moiety -E-.

When D is a group —OC(O)—, the oxygen atom is bonded to the Y′ moiety which is, in turn bonded to the A₁ moiety and the carbon atom is bonded to the Y′ moiety which is, in turn bonded to the moiety -E-.

When F is a group —NR₁₁C(O)—, the carbon atom is bonded to the Y′ moiety which is, in turn bonded to the A₂ moiety and the nitrogen atom is bonded to the Y′ moiety which is, in turn bonded to the moiety -E-.

When F is a group —C(O)NR₁₁—, the nitrogen atom is bonded to the Y′ moiety which is, in turn bonded to the A₂ moiety and the carbon is bonded to the Y′ moiety which is, in turn bonded to the moiety -E-.

When F is a group —C(O)O—, the oxygen atom is bonded to the Y′ moiety which is, in turn bonded to the A₂ moiety and the carbon atom is bonded to the Y′ moiety which is, in turn bonded to the moiety -E-.

When F is a group —OC(O)—, the carbon atom is bonded to the Y′ moiety which is, in turn bonded to the A₂ moiety and the oxygen atom is bonded to the Y′ moiety which is, in turn bonded to the moiety -E-.

The skilled reader will appreciate that in the compound of formula A₁-B-A₂, the values of p, p′, q, q′, r and r′ will be dependent on where the moiety B is attached. Thus, on the phenyl ring on A₁ or A₂ to which B is attached, p+p′ will be 4, q+q′ will be 3 and/or s+r+r′ will be 4.

In one embodiment of the invention, s is 1. In this embodiment, m is preferably 1 and Z is preferably an alkylene group, more preferably, a C₁₋₁₂ alkylene group, more preferably still a C₁₋₁₀ alkylene group, even more preferably a C₁₋₆ or C₁₋₄ alkylene group. More preferably, Z is a CH₂ group.

Preferably, one of R₅ and R₆ is H and the other is selected from H, CO(CH₂)ⱼQ₁ or C═S(NH)(CH₂)ₖQ₂, or R₅, R₆ and the nitrogen to which they are attached together form

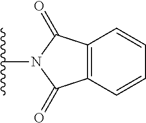

Preferably, L is selected from the following:

### —CH₂NH₂,

### —CH₂NHCOCH₂CH₂COOH,

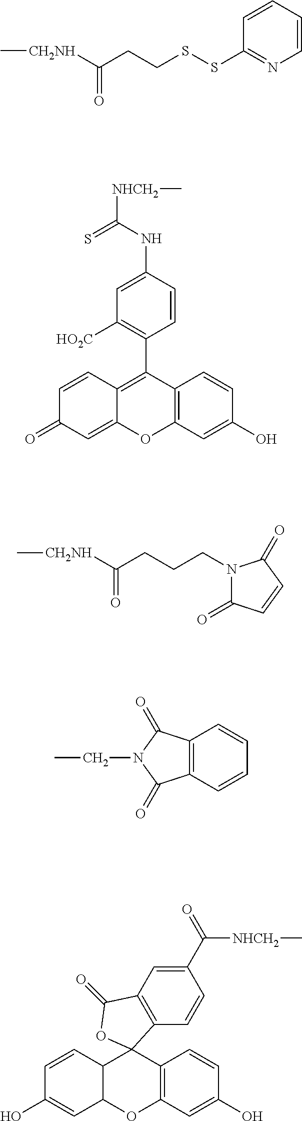

In another embodiment of the invention, s is 0.

Typically A₁ and A₂ are the same.

Preferably each Y′ is the same or different and represents a direct bond or an unsubstituted C₁-C₄ alkyl group, more preferably a direct bond or an unsubstituted C₁-C₂ alkyl group.

Preferably, the straight or branched C₁₋₆ alkyl groups in the definitions of D and F are unsubstituted.

Preferably, the straight or branched C₁₋₆ alkoxy groups in the definitions of D and F are unsubstituted.

Preferably, R₁₁ and R₁₂ each independently represent a hydrogen atom or a straight or branched, unsubstituted C₁₋₆ alkyl group, more preferably a hydrogen atom.

More preferably, D and F are each independently chosen from i) —NR₁₁C(O)—, —C(O)NR₁₁—, —C(O)O—, —OC(O)—; or ii) 5- to 6-membered heteroaryl and 5- to 6-membered non-aromatic heterocyclic groups, which groups are optionally substituted by one, two or three substituents selected from halogen atoms, hydroxyl, NH₂ or straight or branched C₁₋₆ alkyl.

Even more preferably, D and F are each independently chosen from —NR₁₁C(O)—, —C(O)NR₁₁— and unsubstituted 5- to 6-membered heteroaryl groups.

Typically, the unsubstituted 5- or 6-membered heteroaryl groups are unsubstituted 5-membered heteroaryl groups, more preferably unsubstituted 5-membered heteroaryl groups having two or more heteroatoms, which may be the same or different.

Typically, unsubstituted 5-membered heteroaryl groups having two or more heteroatoms, which may be the same or different have two heteroatoms. More preferably, these two heteroatoms are chosen from N and S. Even more preferably, one heteroatom is N and the other S.

Most preferably, D and F are each independently chosen from —NHC(O)—, —C(O)NH— and thiazole groups, in particular 2-thiazole groups.

Typically, the compound of formula A₁-B-A₂ is of formula Xa, Xb, Xc, Xd, Xe or Xf:

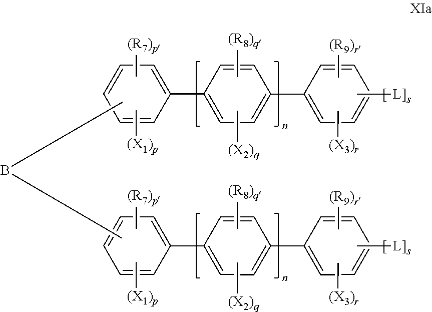

wherein B, R₇, R₈, R₉, X₁, X₂, X₃, n, L and s are as defined above;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′+s equals 5;

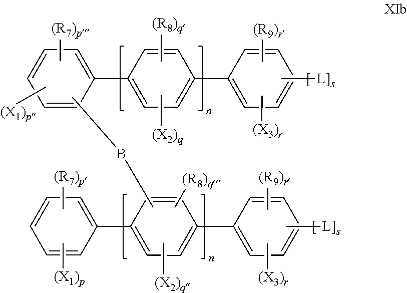

wherein B, R₇, R₈, R₉, X₁, X₂, X₃, n, L and s are as defined above;
p″ is 1, 2, 3, or 4;
p′″ is 0, 1, 2, or 3 where p″+p′″ equals 4;
p is 1, 2, 3, 4 or 5;
p′ is 0, 1, 2, 3 or 4 where p+p′ equals 5;
q″ is 1, 2, or 3;
q′″ is 0, 1, or 2, where q″+q′″ equals 3;
q is 1, 2, 3 or 4;
q′ is 0, 1, 2, or 3 where q+q′ equals 4;
r is 1, 2, 3, 4, or 5;
r′ is 0, 1, 2, 3 or 4 where r+r′+s equals 5;

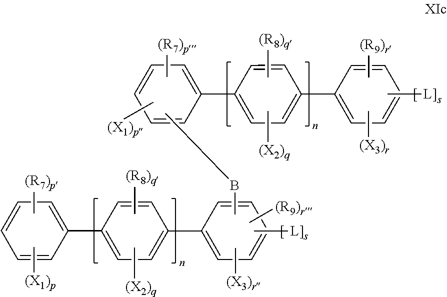

wherein B, R₇, R₈, R₉, X₁, X₂, X₃, n, L and s are as defined above;
p″ is 1, 2, 3, or 4;
p′″ is 0, 1, 2, or 3 where p″+p′″ equals 4;
p is 1, 2, 3, 4 or 5;
p′ is 0, 1, 2, 3 or 4 where p+p′ equals 5;
q is 1, 2, 3 or 4;
q′ is 0, 1, 2 or 3, where q+q′ equals 4;
r″ is 1, 2, 3, or 4;
r′″ is 0, 1, 2, or 3 where r″+r′″+s equals 4;
r is 1, 2, 3, 4, or 5;
r′ is 0, 1, 2, 3 or 4 where r+r′+s equals 5;

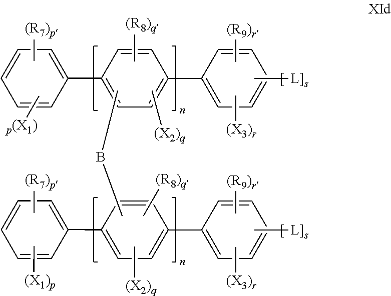

wherein B, R₇, R₈, R₉, X₁, X₂, X₃, n, L and s are as defined above;
p is 1, 2, 3, 4, or 5;
p′ is 0, 1, 2, 3, or 4 where p+p′ equals 5;
q is 1, 2, or 3;
q′ is 0, 1, or 2, where q+q′ equals 3;
r is 1, 2, 3, 4, or 5;
r′ is 0, 1, 2, 3, or 4 where r+r′+s equals 5;

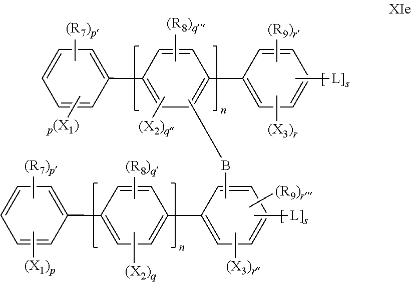

wherein B, R₇, R₈, R₉, X₁, X₂, X₃, n, L and s are as defined above;
p is 1, 2, 3, 4, or 5;
p′ is 0, 1, 2, 3, or 4 where p+p′ equals 5;
q″ is 1, 2, or 3;
q′″ is 0, 1, or 2, where q″+q′″ equals 3;
q is 1, 2, 3 or 4;
q′ is 0, 1, 2, or 3 where q+q′ equals 4;
r″ is 1, 2, 3, or 4;
r′″ is 0, 1, 2, or 3 where r″+r′″+s equals 4;
r is 1, 2, 3, 4, or 5;
r′ is 0, 1, 2, 3 or 4 where r+r′+s equals 5;

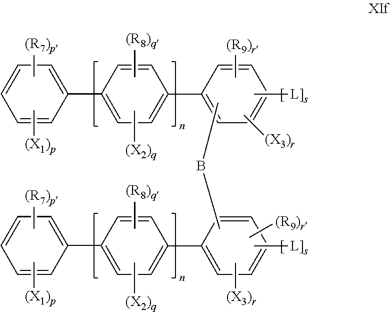

wherein B, R₇, R₈, R₉, X₁, X₂, X₃, n, L and s are as defined above;
p is 1, 2, 3, 4, or 5;
p′ is 0, 1, 2, 3, or 4 where p+p′ equals 5;
q is 1, 2, 3, or 4;
q′ is 0, 1, 2, or 3 where q+q′ equals 4;
r is 1, 2, 3, or 4;
r′ is 0, 1, 2, or 3 where r+r′+s equals 4.

Preferably, the compound of formula A₁-B-A₂ is of formula XIa and s is 0 and is represented by the formula,

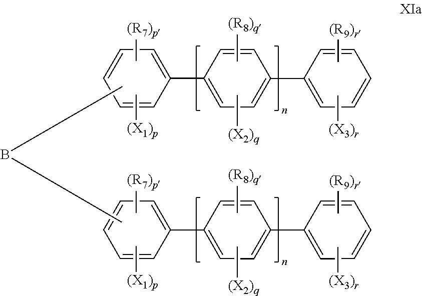

wherein B, R₇, R₈, R₉, X₁, X₂, X₃, n, L and s are as defined above;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′ equals 5.

More preferably, the compound of formula A₁-B-A₂ is of formula,

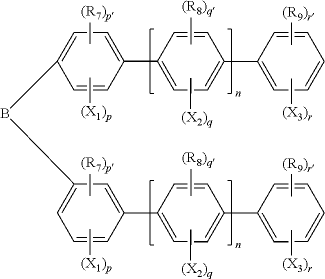

wherein B, R₇, R₈, R₉, X₁, X₂, X₃, n, L and s are as defined above;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′ equals 5,
   formula

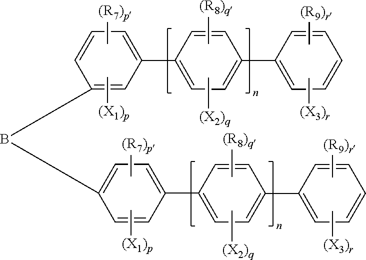

wherein B, R₇, R₈, R₉, X₁, X₂, X₃, n, L and s are as defined above;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′ equals 5
   or formula

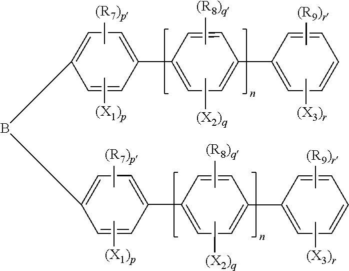

wherein B, R₇, R₈, R₉, X₁, X₂, X₃, n, L and s are as defined above;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′ equals 5.

Typically, Y′ is a direct bond or an unsubstituted C₁-C₄ alkylene group.

Typically, when E is a direct bond, the moiety —Y′—Y′— between the moieties D and E is a C₁-C₈ alkylene group, preferably a C₂-C₈ alkylene group, more preferably a C₂-C₆ alkylene group.

Even more preferably, D is —NHC(O)—, E is a —S—S— group and F is —C(O)NH—; D is —NHC(O)—, E is a direct bond and F is —C(O)NH—; or D and F are 2-thiazole groups and E is a direct bond. Thus, B is —Y—NHC(O)—Y′S—S—Y′—C(O)NH—Y′—, —Y′—NHC(O)—Y′—Y′—C(O)NH—Y′—or

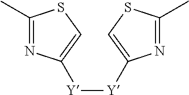

wherein Y′ is as defined above.

Most preferably of all, B is chosen from

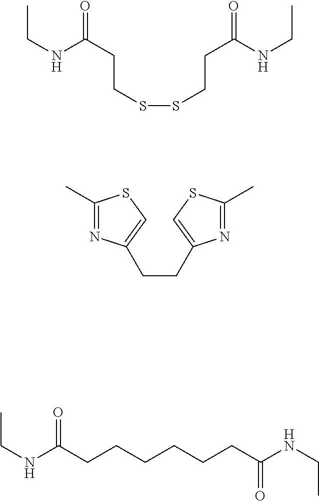

Preferably, A₁ and A₂ are the same.

More preferably, the compound of formula A₁-B-A₂ is represented by the formulae:

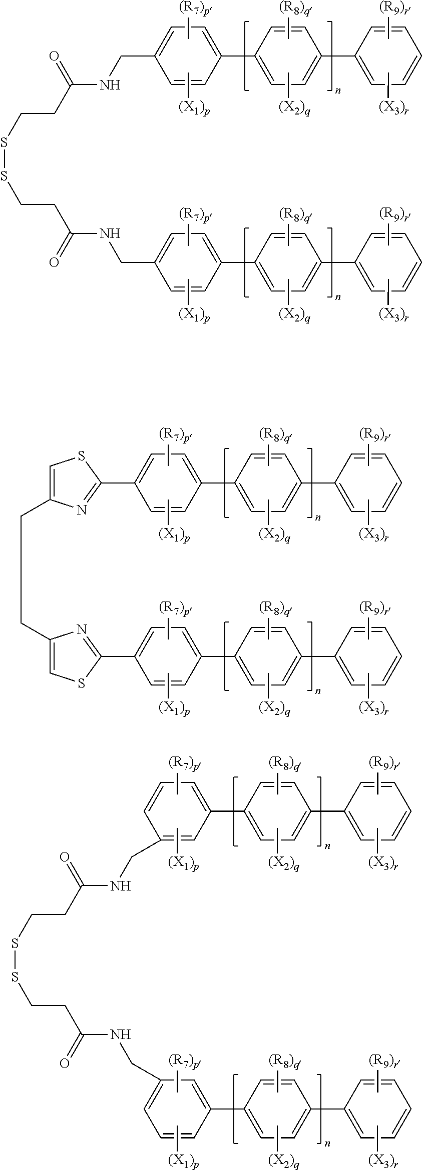

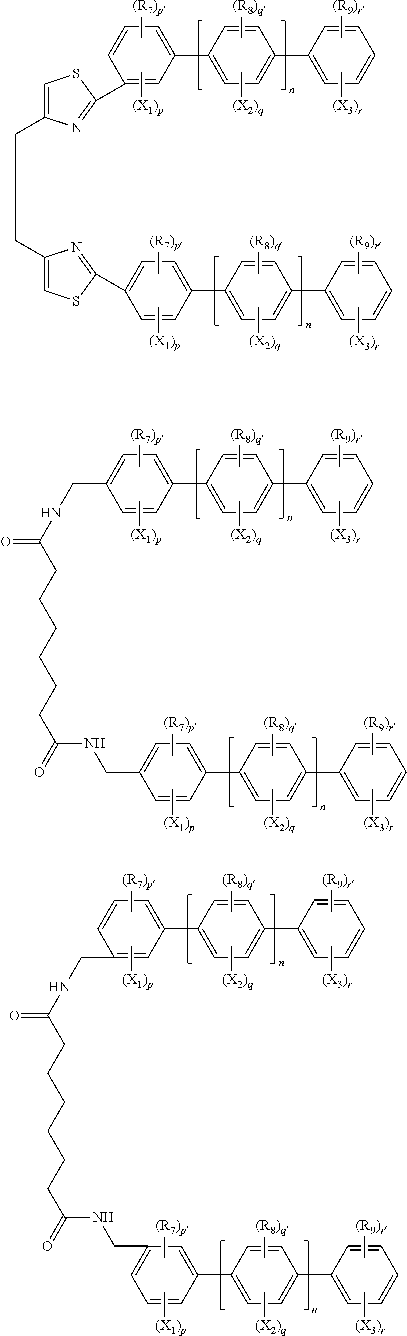

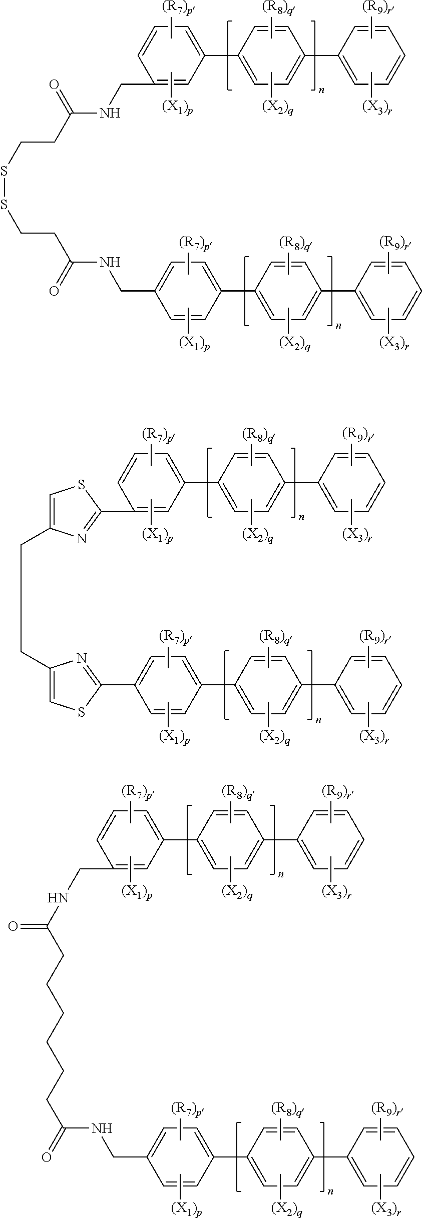

wherein R₇, R₈, R₉, X₁, X₂, X₃, and n are as defined above;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′ equals 5.

Even more preferably, the compound of formula A₁-B-A₂ is represented by the formulae:

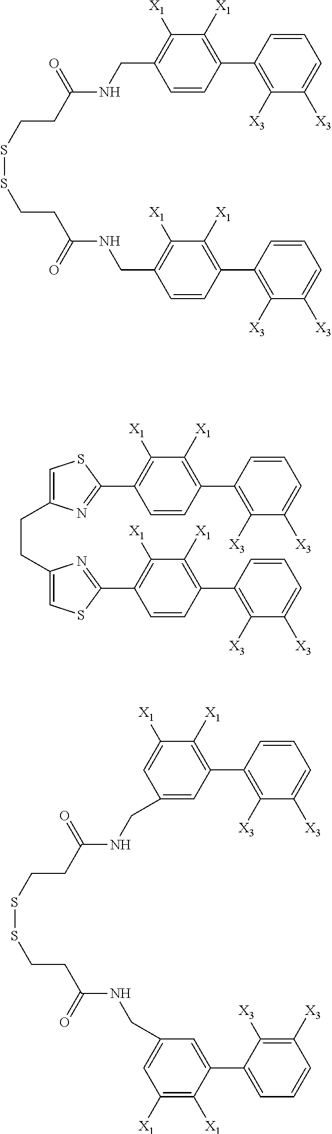

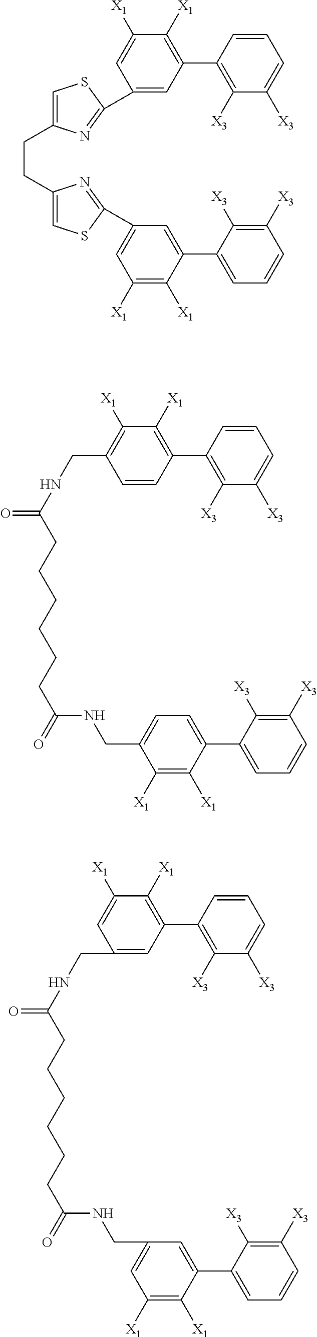

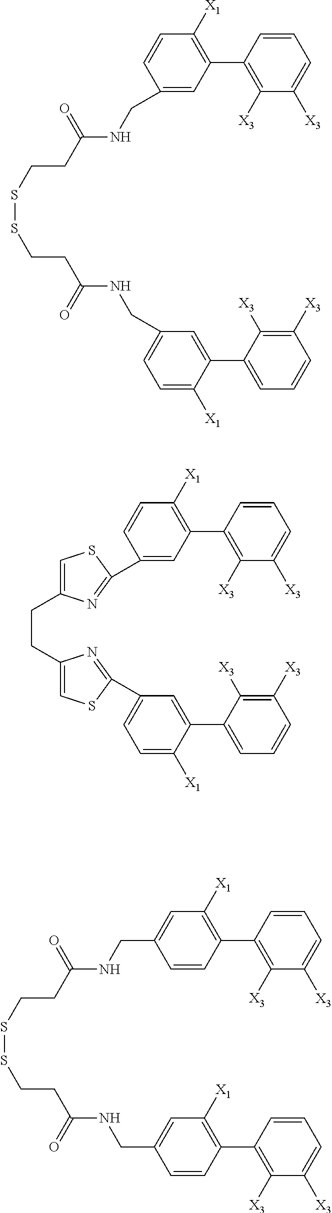

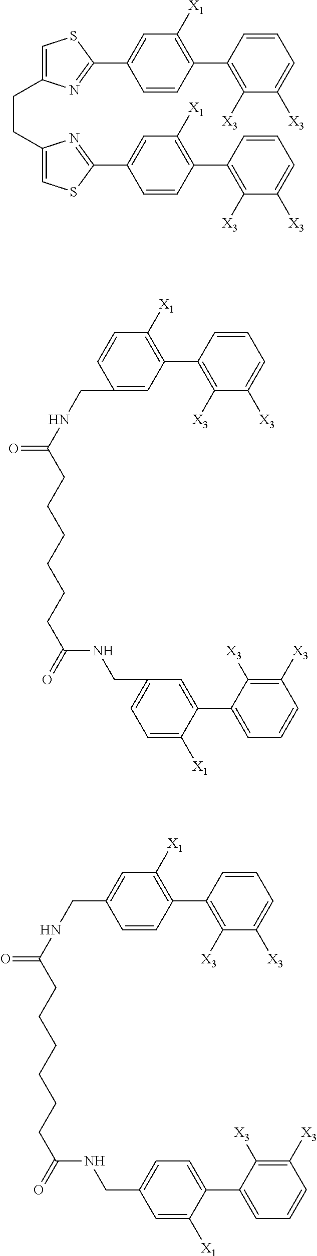

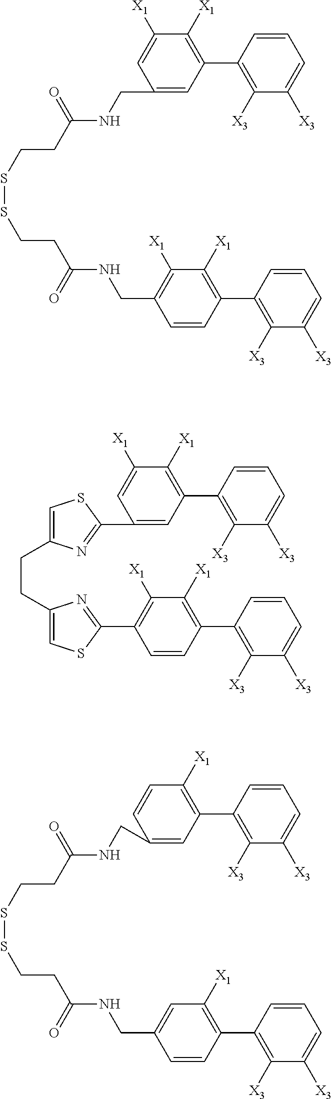

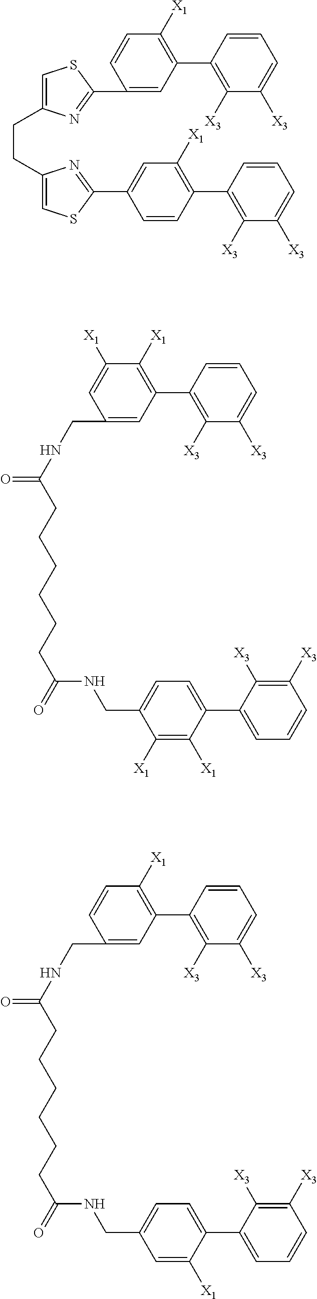

wherein X₁ and X₃ are as defined above.

Specific examples of the compound of formula A₁-B-A₂ are:

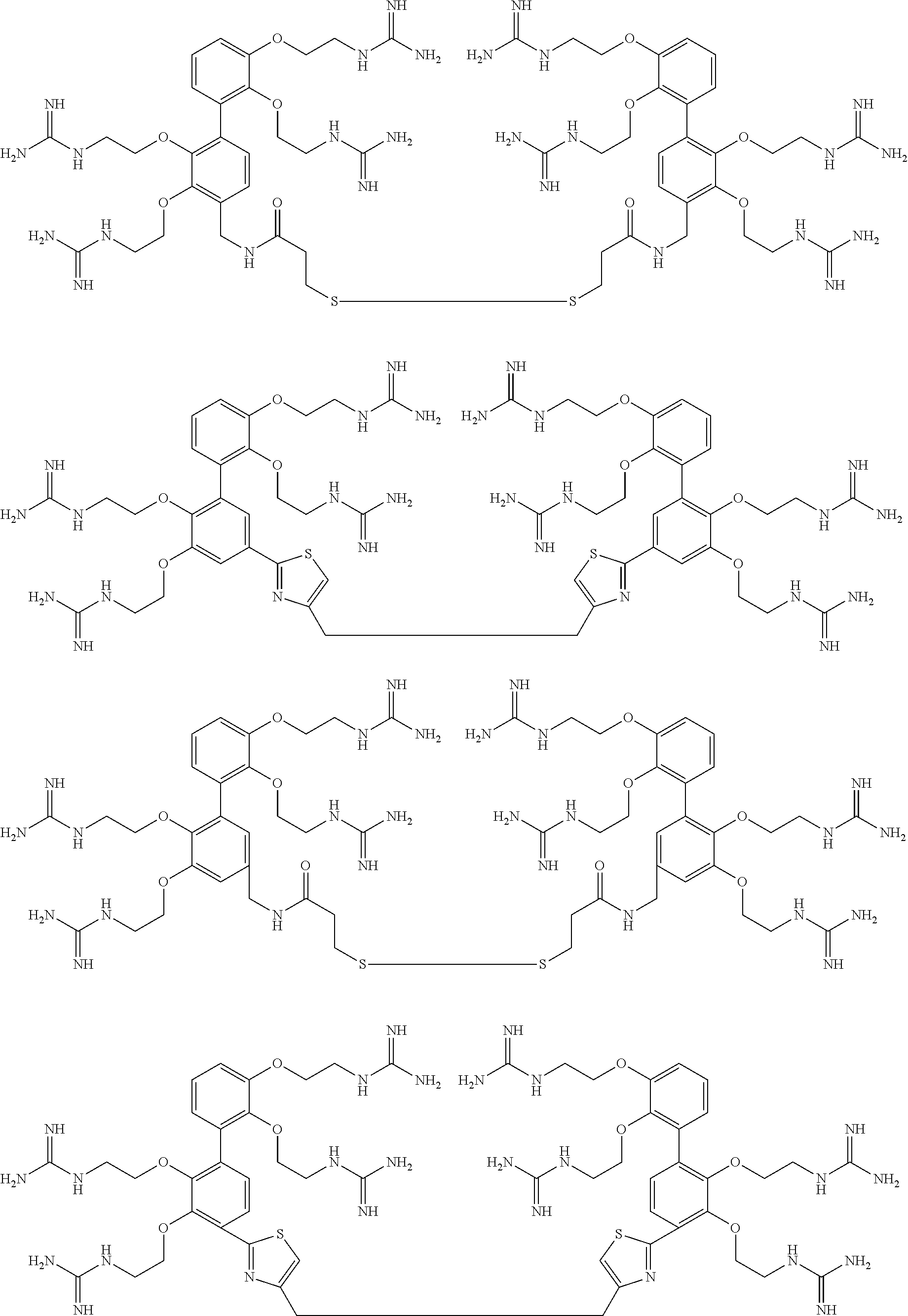

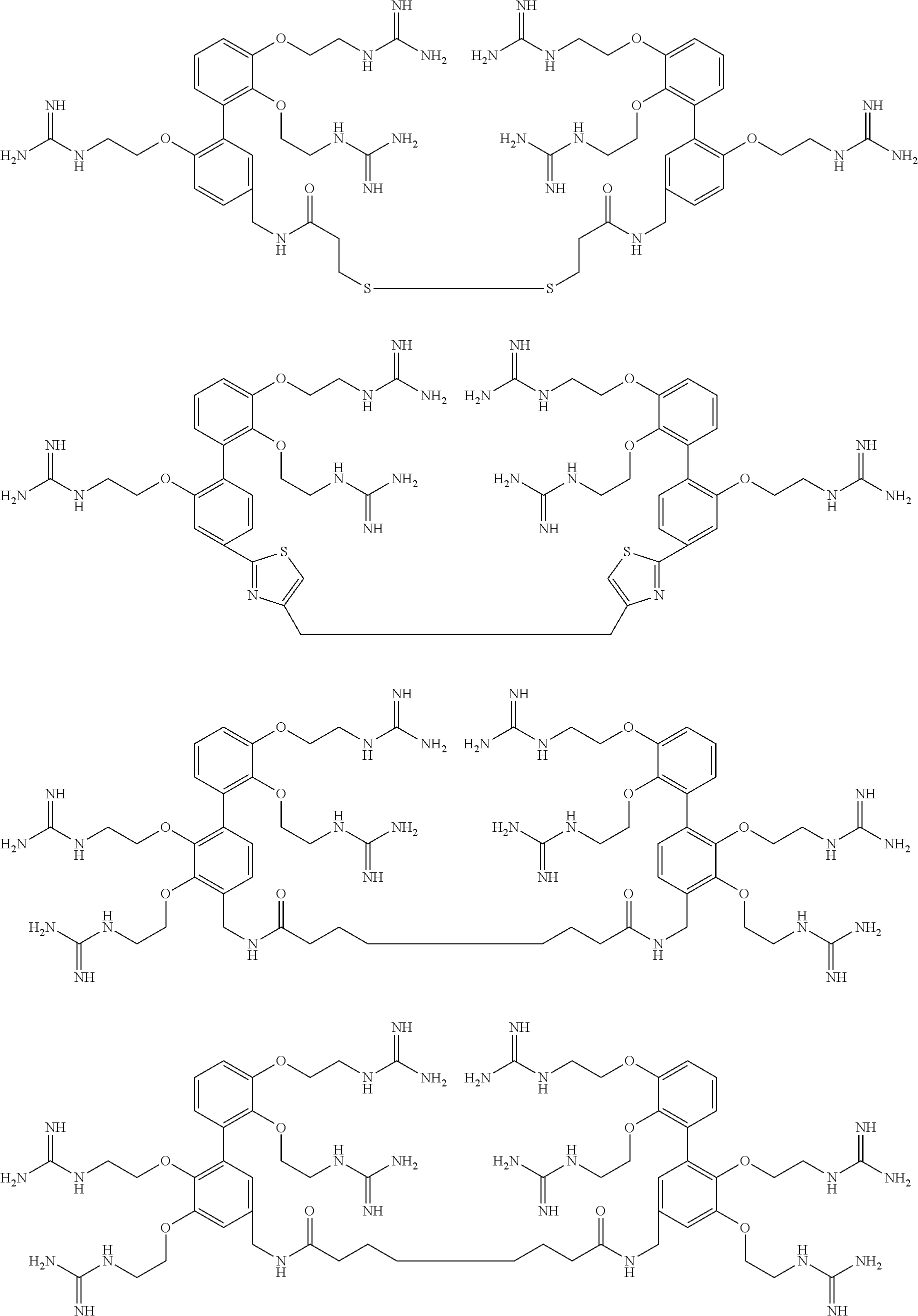

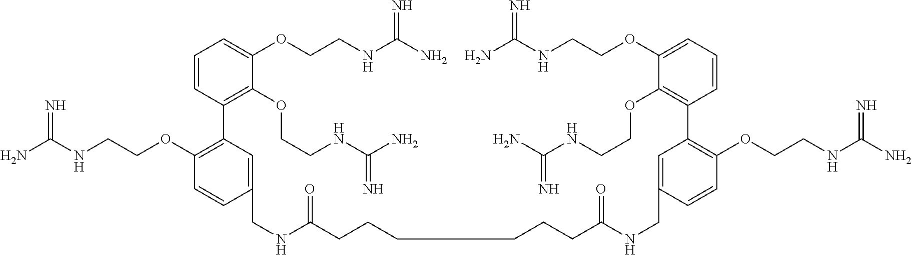

Compounds of formula A₁-B-A₂ can be prepared by methods known to the skilled person in the art. In practice, this will involve reacting a functionalised derivative of a compound of formula A₁ with a functionalised derivative of a compound of formula A₂ under suitable conditions, where a reactive group on the functionalised derivative of A₁ reacts with a group on the functionalised derivative of A₂ to form a compound of A₁-B-A₂. The person skilled in the art can easily select functionalised derivatives of A₁ and A₂ that would react together to form a compound of A₁-B-A₂.

For example, where D or F is —NHC(O)—, compounds of formula A₁-B-A₂ can be prepared by an amidation reaction of a compound A₁ functionalised with an amine group, with a compound of formula A₂ functionalised with an acid chloride group. Alternatively, where D or F is —C(O)O—, compounds of formula A₁-B-A₂ can be prepared by an esterification reaction of a compound A₁ functionalised with an hydroxy group, with a compound of formula A₂ functionalised with an acid chloride group, or a carboxylic acid group.

In a specific example, where D is —NHC(O)—, E is a —S—S— group and F is —C(O)NH—, said process for the production of compounds of formula A₁-B-A₂ comprises reacting a compound of formula A₁-Y′—NHC(O)—Y′—SH with a compound of formula LG₈-S—Y′—C(O)NH—Y′-A₂, where A₁, A₂, and Y′ are as defined above and LG₈ is a leaving group.

LG₈ is typically any group that is a good leaving group in a disulfide bond forming reaction. Those of skill in the art will easily be able to select appropriate leaving groups. LG₈ is preferably a —S-2-pyridyl group.

Alternatively, where D is —NHC(O)—, E is a —S—S— group and F is —C(O)NH—, said process for the production of compounds of formula A₁-B-A₂ comprises reacting a compound of formula A₁-Y′—NHC(O)—Y′—SH with a compound of formula HS—Y′—C(O)NH—Y′-A₂ under oxidative conditions, where A₁, A₂, and Y′ are as defined above.

Compounds of formula A₁-Y—NHC(O)—Y′—SH can be prepared by known methods in the art and are typically prepared by reducing a compound of formula A₁-Y′—NHC(O)—Y′—S-LG₈. Alternatively, compounds of formula A₁-Y—NHC(O)—Y′—SH are prepared by treating a compound of formula A₁-Y—NHC(O)—Y′—S—CPh₃ with AgNO₃ in the presence of acid, for example in the presence of trifluoroacetic acid (TFA). Compounds of formula A₁-Y—NHC(O)—Y′—S—CPh₃ can be prepared by known methods in the art and are typically prepared by treating compounds of formula A₁-Y′—NH₂ with compounds of formula LG₉-C(═O)—Y′—S—CPh₃, where LG₉ is a leaving group.

LG₉ is typically any group that is a good leaving group in a nucleophilic substitution reaction at a carbonyl group. Those of skill in the art will easily be able to select appropriate leaving groups. Examples include —OH, —OTs, —OMs, or a halogen atom. When LG₉ is —OH, one or more coupling agents or catalysts are typically employed, for example N-Ethyldiisopropylamine (DIEA) and/or 2-(1H-7-Azabenzotriazol-1-yl)-1,1,3,3-tetramethyl uronium hexafluorophosphate Methanaminium (HATU).

Compounds of formula A₁-Y′NH₂ can be prepared by known methods, or, for example, by analogous processes to those described in WO-A-05123676 and Rebstock, et al, ChemBioChem 2008, 9, 1-11

Compounds of formulae A₁-Y—NHC(O)—Y′—S-LG₈ and A₂-Y′—NHC(O)—Y′—S-LG₈ can be prepared by known methods, or, for example, by analogous processes to those described in WO-A-05123676 and Rebstock, et al, ChemBioChem 2008, 9(11):1787-1796.

In a further specific example, where B is -D-Y′-E-Y′-F- (i.e. the Y′ groups between A₁ and D, and between F and A₂ both represent direct bonds), D and F are 2-thiazole groups, and E is a direct bond, said process for the production of compounds of formula A₁-B-A₂ comprises reacting a compound of formula A₁-C(═S)NH₂ with a compound of formula A₂-C(═S)NH₂, in the presence of a compound of formula Br—CH₂—C(═O)—Y′—Y′—C(═O)—CH₂—Br, where A₁, A₂, and Y′ are as defined above.

Compounds of formulae Br—CH₂—C(═O)—Y′—Y′—C(═O)—CH₂—Br are commercially available or can be prepared using known methods.

Compounds of formulae A₁-C(═S)NH₂ can be prepared by treating compounds of formulae A₁-CN with sodium hydrogen sulphide and diethylamine hydrochloride.

Compounds of formula A₁-CN can be prepared by known methods, or, for example, by analogous processes to those described in WO-A-05123676 and Rebstock, et al, ChemBioChem 2008, 9, 1-11

Compounds of formulae A₂-C(═S)NH₂ and A₂-CN can be prepared in an analogous manner to compounds of formulae A₁-C(═S)NH₂ and A₁-CN as described above.

In a further specific example, where D is —NHC(O)—, E is a direct bond and F is —C(O)NH—, said process for the production of compounds of formula A₁-B-A₂ comprises reacting a compound of formula A₁-Y′—NH₂, a compound of formula H₂N—Y′-A₂ and a compound of LG₉-C(═O)—Y′—Y′—C(═O)-LG₉ where A₁, A₂, and Y′ are as defined above and LG₉ is a leaving group.

Typically, LG₉ is as defined above.

Compounds of formula A₁-Y′—NH₂ can be prepared as described above.

The conjugate (V) of the invention typically comprises a compound of formula A₁-B-A₂ as defined above non-covalently bound to a cargo moiety by hydrophobic interactions, pi-pi interactions, cation-pi interactions, hydrogen bonding, ionic interactions, van der Waal's forces or dipole-dipole interactions.

The present invention further provides a process for preparing a conjugate (W), which process comprises reacting a compound of formula A₁-B-A₂ as defined above or a pharmaceutically acceptable salt thereof, in which s is 1 and L is other than a moiety —(Z)ₘ-phthalimide, wherein Z and m are as defined as above, with a cargo moiety as defined herein.

The present invention further provides a conjugate (W) obtainable by reacting a compound of formula A₁-B-A₂ as defined above or a pharmaceutically acceptable salt thereof, in which s is 1 and L is other than a moiety —(Z)ₘ-phthalimide, wherein Z and m are as defined above, with a cargo moiety as defined herein.

In the conjugate (W), the cargo moiety may be directly or indirectly linked the compound of formula A₁-B-A₂ (which may be referred to as the carrier moiety). In the embodiment wherein the cargo moiety is indirectly linked to the compound of formula A₁-B-A₂, the linkage may be by an intermediary bonding group such as a sulphydryl or carboxyl group or any larger group, all such linking groups are herein referred to as linker moieties as discussed below. Preferably, the carrier and cargo moieties are linked directly.

In the conjugate (W), the compound of formula A₁-B-A₂ may be linked to two or more cargo moieties, which may be the same or different. Typically, one or more cargo moieties are linked to the A₁ group. Typically, one or more cargo moieties are linked to the A₂ group. Alternatively, two or more cargo moieties may be linked to the A₁ group and no cargo moieties linked to the A₂ group or two or more cargo moieties may be linked to the A₂ group and no cargo moieties linked to the A₁ group.

Preferably, in the conjugate of formula (W) the cargo moiety is covalently attached to the L group of the compound of formula A₁-B-A₂. Typically, a reactive group in the cargo moiety reacts with a reactive group in the L moiety of the compound of formula A₁-B-A₂.

Typically, in the formation of the conjugate of formula (W), a nucleophilic group on the cargo moiety (for example an amine, thiol or hydroxy group) displaces a leaving group in the moiety L in the formula A₁-B-A₂. For example, a thiol-containing protein (eg geminin) can react with a compound of formula A₁-B-A₂ in which Q₁ or Q₂ is —S—S-(2-pyridyl) via thiol exchange. Similarly, a nucleophilic moiety such as the 2′-hydroxy group on the taxol or docetaxel molecule can displace a succinimyl, —S—S-(2-pyridyl), iodine, —S—S(O)₂—OMe or —CO—O—N-succininyl group in the moiety L in formula A₁-B-A₂.

Alternatively, in the formation of the conjugate of formula (W), when L is nucleophilic (eg when L is —NH₂), it can react with an electrophilic site in the cargo moiety.

In one preferred embodiment of the conjugate (W), the cargo moiety is directly linked to the carrier moiety.

In another preferred embodiment of the conjugate (W), the cargo moiety is indirectly linked to the carrier moiety by means of a linker moiety. In this embodiment, the cargo moiety comprises a protein, a peptide, an oligonucleotide, a nucleotide, a diagnostic agent, or a drug which is attached to a linker moiety. Typically, a reactive site on the linker group reacts with the moiety L in the formula A₁-B-A₂ as explained above.

Direct linkage may occur through any convenient functional group on the cargo moiety, such as a hydroxy, carboxy or amino group. Indirect linkage will occur through a linking moiety. Suitable linking moieties include bi- and multi-functional alkyl, aryl, aralkyl or peptidic moieties, alkyl, aryl or aralkyl aldehydes acids esters and anhydrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional group on the linker moiety used to form covalent bonds between the compound of formula I and the cargo moiety may be, for example, amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the compound of formula A₁-B-A₂. Alternatively, the compound of formula I and the cargo moiety may be linked by leucine zippers, dimerisation domains, or an avidin/biotin linker.

The cargo moiety can be an oligonucleotide, nucleotide, protein, peptide, diagnostic agent, a drug or a combination thereof.

Preferably the cargo moiety is an oligonucleotide.

Examples of suitable oligonucleotide cargo moieties include genes, gene fragments, sequences of DNA, cDNA, RNA, nucleotides, nucleosides, heterocyclic bases, synthetic and non-synthetic, sense or anti-sense oligonucleotides including those with nuclease resistant backbones etc. or any of the above incorporating a radioactive label, that are desired to be delivered into a cell or alternatively to be delivered from a cell to its exterior. Preferably, the oligonucleotide cargo moiety is a gene or gene fragment.

More preferably the oligonucleotide is RNA, most preferably siRNA.

In a particularly preferred embodiment, the siRNA is an siRNA that targets the cdc25, preferably cdc25A, or cdc7 gene.

In a further particularly preferred embodiment, the siRNA is an siRNA that targets a neuronal cell, a dendritic cell, a macrophage or a stem cell, which can be a human or non-human stem cell.

Examples of suitable protein or peptide cargo moieties include; proteins, peptides, and their derivatives such as: antibodies and fragments thereof; cytokines and derivatives or fragments thereof, for example, the interleukins (IL) and especially the IL-1, IL-2, IL-3, IL-4, IL-5, IL-6, IL-7, IL-8, IL-9, IL-10, IL-11 and IL-12 subtypes thereof; colony stimulating factors, for example granulocyte-macrophage colony stimulating factor, granulocyte-colony stimulating factor (alpha and beta foams), macrophage colony stimulating factor (also known as CSF-1); haemopoietins, for example erythropoietin, haemopoietin-alpha and kit-ligand (also known as stem cell factor or Steel factor); interferons (IFNS), for example IFN-α, IFN-β and IFN-γ; growth factors and bifunctional growth modulators, for example epidermal growth factor, platelet derived growth factor, transforming growth factor (alpha and beta forms), amphiregulin, somatomedin-C, bone growth factor, fibroblast growth factors, insulin-like growth factors, heparin binding growth factors and tumour growth factors; differentiation factors and the like, for example macrophage differentiating factor, differentiation inducing factor (DIF) and leukaemia inhibitory factor; activating factors, for example platelet activating factor and macrophage activation factor; coagulation factors such as fibrinolytic/anticoagulant agents including heparin and proteases and their pro-factors, for example clotting factors VII, VIII, IX, X, XI and XII, antithrombin III, protein C, protein S, streptokinase, urokinase, prourokinase, tissue plasminogen activator, fibrinogen and hirudin; peptide hormones, for example insulin, growth hormone, gonadotrophins, follicle stimulating hormone, leutenising hormone, growth hormone releasing hormone and calcitonin; enzymes such as superoxide dismutase, glucocerebrosidase, asparaginase and adenosine deaminase; vaccines or vaccine antigens such as, for example hepatitis-B vaccine, malaria vaccine, melanoma vaccine and HIV-1 vaccine; transcription factors and transcriptional modulators.

Preferably the protein or peptide is an antibody.

In one preferred embodiment, the cargo moiety is selected from a recombinant antibody, a Fab fragment, a F(ab′)₂ fragment, a single chain Fv, a diabody, a disulfide linked Fv, a single antibody domain and a CDR.

As used herein, the term “CDR″ or “complementary determining region” refers to the hypervariable regions of an antibody molecule, consisting of three loops from the heavy chain and three from the light chain, that together form the antigen-binding site.

By way of example, the antibody may be selected from Herceptin, Rituxan, Theragyn (Pemtumomab), Infliximab, Zenapex, Panorex, Vitaxin, Protovir, EGFR1 or MFE-23.

In one preferred embodiment, the cargo moiety is a genetically engineered fragment selected from a Fab fragment, a F(ab′)₂ fragment, a single chain Fv, or any other antibody-derived format.

Conventionally, the term “Fab fragment” refers to a protein fragment obtained (together with Fc and Fc′ fragments) by papain hydrolysis of an immunoglobulin molecule. It consists of one intact light chain linked by a disulfide bond to the N-terminal part of the contiguous heavy chain (the Fd fragment). Two Fab fragments are obtained from each immunoglobulin molecule, each fragment containing one binding site. In the context of the present invention, the Fab fragment may be prepared by gene expression of the relevant DNA sequences.

Conventionally, the term “F(ab′)₂” fragment refers to a protein fragment obtained (together with the pFc′ fragment) by pepsin hydrolysis of an immunoglobulin molecule. It consists of that part of the immunoglobulin molecule N-terminal to the site of pepsin attack and contains both Fab fragments held together by disulfide bonds in a short section of the Fc fragment (the hinge region). One F(ab′)₂ fragment is obtained from each immunoglobulin molecule; it contains two antigen binding sites, but not the site for complement fixation. In the context of the present invention, the F(ab′)₂ fragment may be prepared by gene expression of the relevant DNA sequences.

As used herein, the term “Fv fragment” refers to the N-terminal part of the Fab fragment of an immunoglobulin molecule, consisting of the variable portions of one light chain and one heavy chain. Single-chain Fvs (about 30 KDa) are artificial binding molecules derived from whole antibodies, but which contain the minimal part required to recognise antigen.

In another preferred embodiment, the cargo moiety is a synthetic or natural peptide, a growth factor, a hormone, a peptide ligand, a carbohydrate or a lipid.

The cargo moiety can be designed or selected from a combinatorial library to bind with high affinity and specificity to a target antigen. Typical affinities are in the 10⁻⁶ to 10⁻¹⁵ M K_{d} range. Functional amino acid residues present in the cargo moiety may be altered by site-directed mutagenesis where possible, without altering the properties of the cargo moiety. Examples of such changes include mutating any free surface thiol-containing residues (cysteine) to serines or alanines, altering lysines and arginines to asparagines and histidines, and altering serines to alanines.

In another preferred embodiment the cargo moiety is protein A, a bacterially derived protein that binds strongly to conventional antibodies.

Previous studies have demonstrated that a fusion protein containing the protein transduction domain of HIV-1 TAT and the B domain of staphylococcal protein A can be used to internalise antibodies into mammalian cells [Mie et al, Biochemical and Biophysical Research Communications 310 (2003); 730-734].

Preferably, in the conjugate (W) the compound of formula A₁-B-A₂ is linked to commercially available (natural) protein A via a lysine NH₂ group of protein A.

In one particularly preferred embodiment of the invention, the conjugate (W) is the reaction product of a protein (such as for example, protein A) and a compound of formula A₁-B-A₂ as shown above wherein L is (Z)ₘNR₅R₆ where Z is a hydrocarbyl group, as defined above, and m is 0 or 1; where R₅ and R₆ are each independently H, CO(CH₂)ⱼQ₁ or C═S(NH)(CH₂)ₖQ₂ where j and k are defined above and Q₁ and Q₂ are each independently

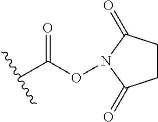

In an alternative preferred embodiment, a cysteine residue may be engineered into the protein to allow conjugation in the conjugate of formula (W) to said compound of formula A₁-B-A₂. Further details on the preparation of cysteine modified proteins may be found in Neisler et al [Bioconjugate Chem. 2002, 13, 729-736].

The diagnostic agent can be nonbiological, for example a microbead. Appropriate processes for preparing a conjugate of a compound of formula A₁-B-A₂ and a nonbiological diagnostic agent such as a microbead are familiar to those of skill in the art.

In a further embodiment of the invention, the cargo moiety is a drug. In this embodiment, the conjugate can be described as a delivery system. Preferably, the delivery system is therapeutically active in its intact state.

Drugs are typically selected from cytotoxic agents such as doxorubicin, methotrexate and derivatives thereof, anti-neoplastic agents, anti-hypertensives, cardioprotective agents, anti-arrhythmics, ACE inhibitors, anti-inflammatory's, diuretics, muscle relaxants, local anaesthetics, hormones, cholesterol lowering drugs, anti-coagulants, anti-depressants, tranquilizers, neuroleptics, analgesics such as a narcotic or anti-pyretic analgesics, anti-virals, anti-bacterials, bacteristatic and bactericidal agents, anti-fungals, anthelminthics and other agents effective against infective agents including unicellular pathogens; bacteriostats, CNS active agents, anti-convulsants, anxiolytics, antacids, narcotics, antibiotics including lantibiotics, respiratory agents, anti-histamines, immunosuppressants, immunoactivating agents, nutritional additives, anti-tussives, emetics and anti-emetics, carbohydrates, glycosoaminoglycans, glycoproteins and polysaccharides, lipids, for example phosphatidyl-ethanolamine, phosphtidylserine and derivatives thereof, sphingosine, steroids, vitamins, small effector molecules such as noradrenalin, alpha adrenergic receptor ligands, dopamine receptor ligands, histamine receptor ligands, GABA/benzodiazepine receptor ligands, serotonin receptor ligands, leukotrienes and triodothyronine.

More preferably, the drug moiety is derived from a cytotoxic drug.

More preferably, the drug moiety is selected from DNA damaging agents, anti-metabolites, anti-tumour antibiotics, natural products and their analogues, dihydrofolate reductase inhibitors, pyrimidine analogues, purine analogues, cyclin-dependent kinase inhibitors, thymidylate synthase inhibitors, DNA intercalators, DNA cleavers, topoisomerase inhibitors, anthracyclines, vinca drugs, mitomycins, bleomycins, cytotoxic nucleosides, pteridine drugs, diynenes, podophyllotoxins, platinum containing drugs, differentiation inducers and taxanes.

Even more preferably, the drug moiety is selected from methotrexate, methopterin, dichloromethotrexate, 5-fluorouracil, 6-mercaptopurine, tri-substituted purines such as olomoucine, roscovitine and bohemine, flavopiridol, staurosporin, cytosine arabinoside, melphalan, leurosine, actinomycin, daunorubicin, doxorubicin, mitomycin D, mitomycin A, caminomycin, aminopterin, tallysomycin, podophyllotoxin (and derivatives thereof), etoposide, cisplatinum, carboplatinum, vinblastine, vincristine, vindesin, paclitaxel, docetaxel, taxotere retinoic acid, butyric acid, acetyl spermidine, tamoxifen, irinotecan, camptothecin, atorvastatin, clopidogrel, enoxaparin, celecoxib, omeprazole, esomeprazole, fexofenadine, quetiapine, metoprolol and budesonide.

In one preferred embodiment of the conjugate (W), the drug moiety is directly linked to the carrier moiety.

In another preferred embodiment of the conjugate (W), the drug moiety is indirectly linked to the carrier moiety by means of a linker moiety.

In another preferred embodiment, each carrier moiety is linked or non-covalently bound to more than one drug moiety.

In one preferred embodiment, where each carrier moiety is linked or non-covalently bound to more than one drug moiety, the drug moieties are different.

In a further preferred embodiment of the invention, the delivery system may further comprise a targeting moiety. The targeting moiety is capable of directing the delivery system to the specific cell type to which it is preferable for the drug moiety to function. Thus, the targeting moiety acts as an address system biasing the body's natural distribution of drugs or the delivery system to a particular cell type. Typically, the targeting moiety is attached to the drug moiety. In the conjugate (W), the targeting moiety may be attached to the drug moiety or alternatively to the carrier moiety.

In one preferred embodiment of the conjugate (W), the targeting moiety is directly linked to the carrier moiety.

In another preferred embodiment of the conjugate (W), the targeting moiety is indirectly linked to the carrier moiety by means of a linker moiety.

Direct linkage may occur through any convenient functional group on the targeting moiety, such as a hydroxy, carboxy or amino group. Indirect linkage will occur through a linking moiety. Suitable linking moieties include bi- and multi-functional alkyl, aryl, aralkyl or peptidic moieties, alkyl, aryl or aralkyl aldehydes acids esters and anhydrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds to the targeting moiety may be two or more of, e.g., amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the targeting moiety. Alternatively, the targeting moiety may be linked by leucine zippers, dimerisation domains, or an avidin/biotin linker.

Another aspect of the invention relates to a pharmaceutical composition comprising a compound or conjugate of the invention admixed with one or more pharmaceutically acceptable diluents, excipients or carriers. Even though the compounds and conjugates of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

In all aspects of the present invention previously discussed, the invention includes, where appropriate all enantiomers and tautomers of compounds of the invention. The man skilled in the art will recognise compounds that possess optical properties (one or more chiral carbon atoms) or tautomeric characteristics. The corresponding enantiomers and/or tautomers may be isolated/prepared by methods known in the art.

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F, and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

The present invention also includes the use of solvate forms of the compounds of the present invention. The terms used in the claims encompass these forms.

The invention furthermore relates to the compounds and/or conjugates of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

A person of ordinary skill in the art can easily determine an appropriate dose of one of the compositions of the invention to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight. In an exemplary embodiment, one or more doses of 10 to 150 mg/day will be administered to the patient.

In a particularly preferred embodiment, the one or more compounds and/or conjugates of the invention are administered in combination with one or more other therapeutically active agents, for example, existing drugs available on the market. In such cases, the compounds of the invention may be administered consecutively, simultaneously or sequentially with the one or more other therapeutically active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in cells which would have been otherwise responsive to initial chemotherapy with a single agent.

By way of example, numerous combinations are used in current treatments of cancer and leukaemia. A more extensive review of medical practices may be found in “Oncologic Therapies” edited by E. E. Vokes and H. M. Golomb, published by Springer.

The present invention also provides use of a compound or conjugate of the invention in the manufacture of a medicament for use delivering a drug to a patient transdermally.

The present invention also provides a skin patch which comprises a compound or conjugate of the invention and a pharmaceutically acceptable carrier or diluent.

The following Examples illustrate the invention.

### EXAMPLES

All starting materials were either commercially available or synthesised by methods reported in the literature. ¹H and ¹³C spectra were recorded on a Bruker AMX-300 spectrometer. Chemical shifts are reported as ppm relative to TMS as internal standard. Mass spectra were recorded on either a VG ZAB SE spectrometer (EI, FAB) or a Gilson-Finningan AQA LC-mass spectrometer using C-18 column (Hypersil BDS 100×4.6 mm, 5 μm). Microanalysis was carried out by the Analytical Services Section, Department of Chemistry, University College London. Purification was by reverse-phase HPLC (Gilson) using preparative C-18 columns (Hypersil PEP 100×21 mm, 5 μm). Melting points were determined on a Gallenkamp melting point apparatus and are uncorrected. IR spectra were recorded on a Perkin-Elmer Spectrum One series FT-IR spectrophotometer. The microwave experiments were run on a Biotage Initiator 60 microwave.

### Example 1

### Synthesis of SMOC Carrying 6 Guanidine Moieties

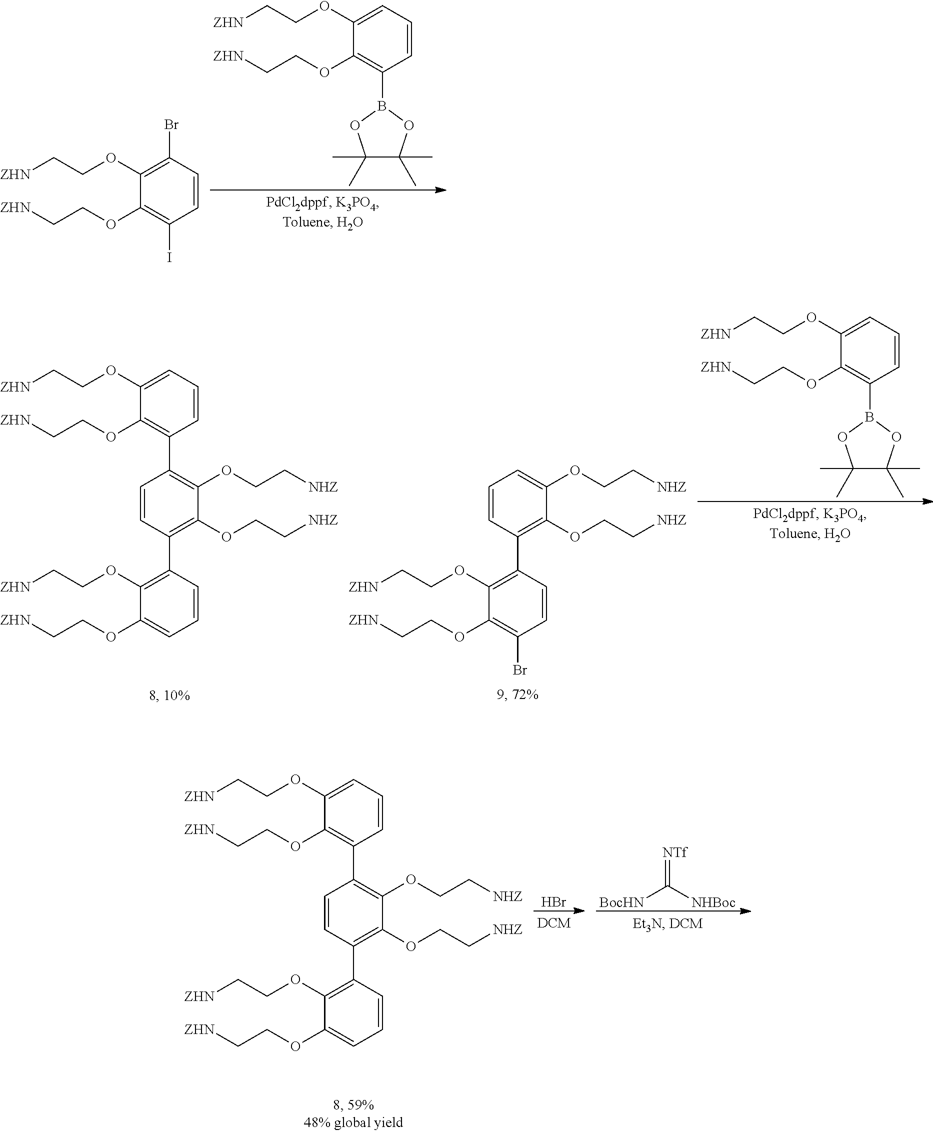

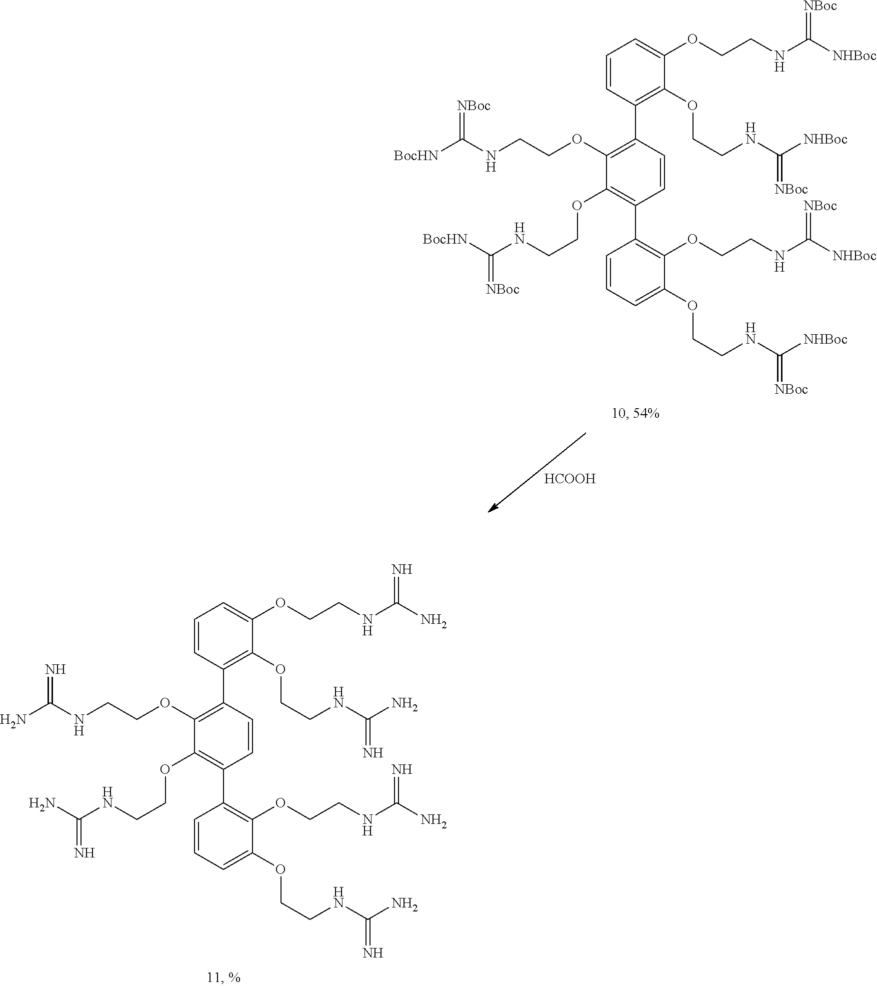

### 6-SMoC—NHZ 8

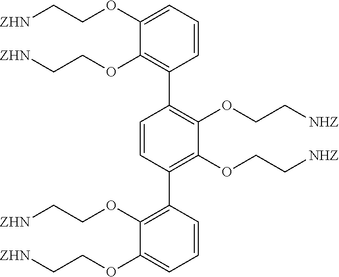

A degassed mixture of dibenzyl 2,2′-(3-bromo-6-iodo-1,2-phenylene)bis(oxy)bis(ethane-2,1-diyl)dicarbamate (1 g, 1.5 mmol), dibenzyl 2,2′-(3-(4,4,5,5-tetramethyl-1,3,2-dioxaborolan-2-yl)-1,2-phenylene)bis(oxy)bis(ethane-2,1-diyl)dicarbamate (1.77 g, 3 mmol), PdCl₂dppf.CH₂Cl₂ (61 mg, 0.075 mmol), potassium phosphate (0.69 g, 3 mmol), toluene (10 mL) and water (1 mL) was heated at 100° C. for 3 h. The reaction mixture was diluted in EtOAc (25 mL) and water (25 mL) was added. The layers were separated and the aqueous layer was extracted with EtOAc (3×25 mL). The combined organic layers were dried over MgSO₄, filtered and concentrated under vacuum. The residue was then purified by flash chromatography using cyclohexane/EtOAc (1/1) as eluent to afford 8 (0.21 g, 10% Yield) as a yellow oil and benzyl 2,2′, 2″, 2′″-(4-bromobiphenyl-2,2′,3,3′-tetrayl)tetrakis(oxy)tetrakis(ethane-2,1-diyl)tetracarbamate 9 (1.09 g, 1.08 mmol).

A degassed mixture of 9 (1.09 g, 1.08 mmol), dibenzyl 2,2′-(3-(4,4,5,5-tetramethyl-1,3,2-dioxaborolan-2-yl)-1,2-phenylene)bis(oxy)bis(ethane-2,1-diyl)dicarbamate (0.94 g, 1.6 mmol), PdCl₂dppf.CH₂Cl₂ (40 mg, 0.05 mmol), potassium phosphate (0.46 g, 2 mmol), toluene (7.5 mL) and water (0.75 mL) was heated at 100° C. overnight. The reaction mixture was diluted in EtOAc (25 mL) and water (25 mL) was added. The layers were separated and the aqueous layer was extracted with EtOAc (3×25 mL). The combined organic layers were dried over MgSO₄, filtered and concentrated under vacuum. The residue was then purified by flash chromatography using cyclohexane/EtOAc (1/1) as eluent to afford 8 (0.88 g, 59% yield) as a yellow oil. The global yield was 48%. There is 0.66 g 8.

### 6G-SMoC-GBoc 10

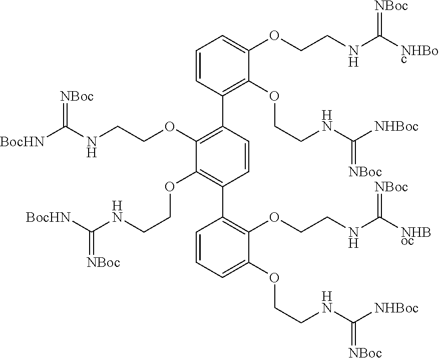

6-SMoC-NHZ 8 (0.15 g, 0.11 mmol) was dissolved in DCM (4 mL) and HBr (30% in acetic acid, 1 mL) was added dropwise. After stirring at room temperature for 1.5 h, water (25 mL) was added to the mixture, the layers were separated and the aqueous layer was washed with DCM (2×25 mL). The water was then removed under vacuum and the crude tetra-amine was carefully dried under vacuum for several hours.

The resulting solid was suspended in DCM (5 mL) and DMA (1 mL) and N,N-di-boc-N′-trifluoromethanesulfonyl-guanidine (0.25 g, 0.63 mmol) were added. The mixture was stirred overnight at room temperature, diluted with DCM (20 mL) then washed with 2M NaHSO₄ 2M (25 mL) followed by sat. aq. NaHCO₃ (25 mL) and brine (25 mL). The organic layer was dried over MgSO₄, filtered and concentrated under vacuum. The residue was purified by flash chromatography using cyclohexane/EtOAc (85:15 to 65:35) as eluent to afford the title compound 10 (0.12 g, 54% yield) as a white solid.

### 6G-SMoC 11

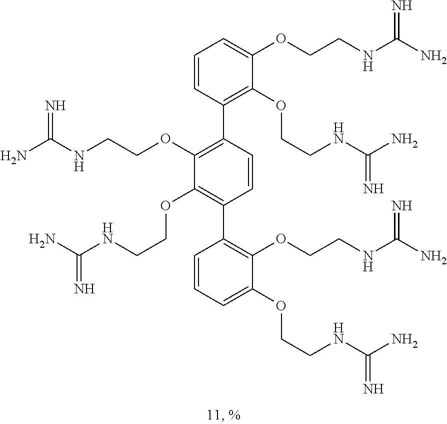

To the Boc compound 10 (90 mg, 0.049 mmol) was added formic acid (1 mL) and the reaction stirred overnight at r.t., the temperature was raised to 50° C. for 1 hour. The formic acid was removed on the rotary evaporator and the residue freeze dried from 10 mL of water. This gave 40 mg product. Maldi MS indicated the presence of product with no evident impurities.

MS (MALDI) 859 [M+Na]⁺. 837.6 [MH]⁺.

### Example 2

### Analysis of Binding of 4G-SMOC Compound to siRNA

An Agarose electrophoresis gel shift experiment was carried out to analyse the strength of the binding between the 4G-SMOC compound shown below and siRNA targeted to the cdc25A and cdc7 genes.

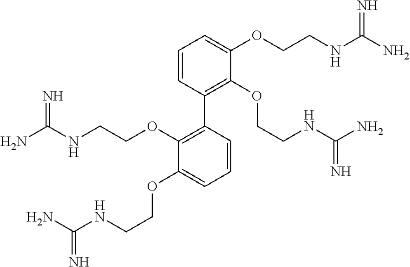

The experiment was carried out using a protocol substantially as described in Lundberg, et al, FASEB J., 2007, 21, 2664-2671, the entirety of which is incorporated herein by reference.

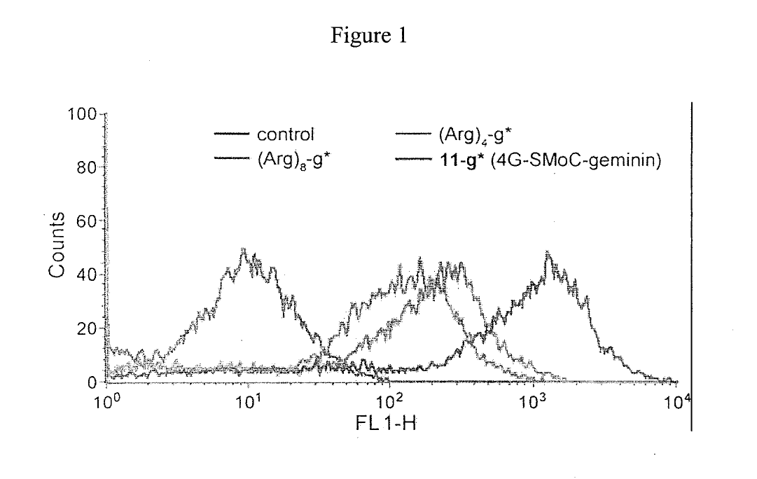

The results of this experiment are shown as FIG. 1. These results show that the binding of siRNA with the 4G-SMOC compound is similar to that reported for cell penetrating peptides (CPPs) in Lundberg, et al, 2007.

### Example 3

### Analysis of Binding of 6G-SMOC Compound to siRNA

An isothermal calorimetry experiment (ITC) was carried out to analyse the strength of the binding between the 6G-SMOG compound shown below and siRNA targeted to the cdc25A and cdc7 genes.

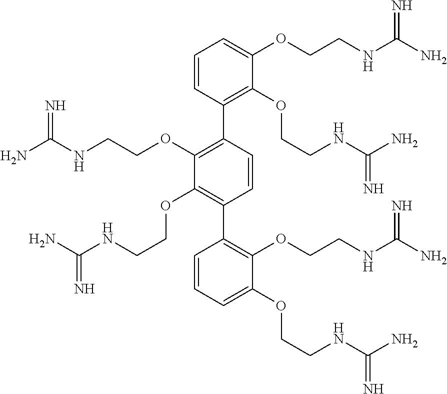

6G-SMOC at a concentration of 250 μM was loaded into the syringe of a Microcal VP-ITC calorimeter (450 μl), and the cell (1.8 ml) filled with GAPD siRNA (Dharmacon RNAi Technologies) at a concentration of 3.5 μM. A total of 37 injections of 8 μl each were made at 4 minute intervals to ensure total saturation of the siRNA. The binding curve was plotted using Origin 6.0 (Microcal) and the binding constants calculated.

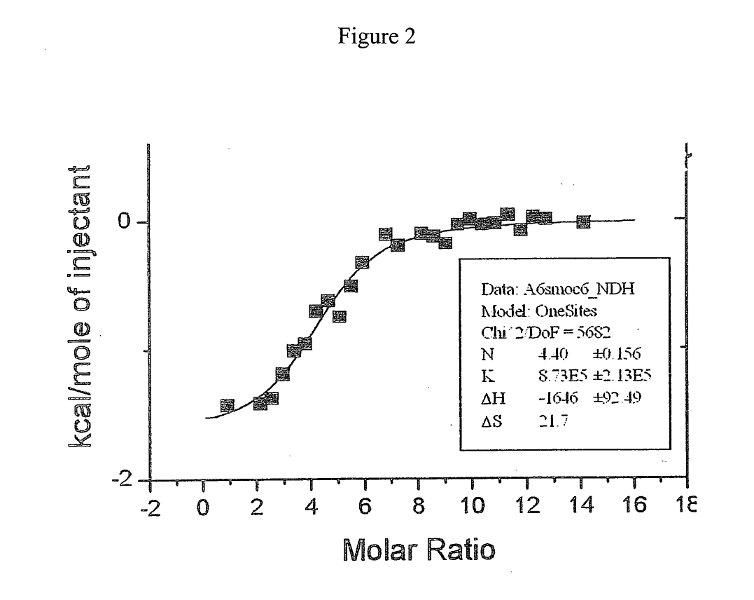

The results of this experiment are shown as FIG. 2. A Kd of approximately 80 μM was obtained with a stoichiometry of 4:1 6G-SMOC:siRNA.

### Example 4

### Observation of Cellular Uptake Via Live Microscopy (Richard, et al, 2003, J. Biol. Chem. 278, 585-590)

For detection of different SMOC-Flu-siRNA uptake into live cells, exponentially growing cells (WI-38 HDF) are cultured on glass coverslips. Cells are washed in PBS, and incubated with fresh medium containing the siRNA-SMOC complex at several concentrations. Any dye markers are added at this stage. Coverslips are washed extensively in PBS, placed in a plate containing medium without Red Phenol (Gebco) and observed via live confocal microscopy (MP-UV, Leica Microsystems GmbH, Wetzlar, Germany) using 40× and 60× water immersion objectives.

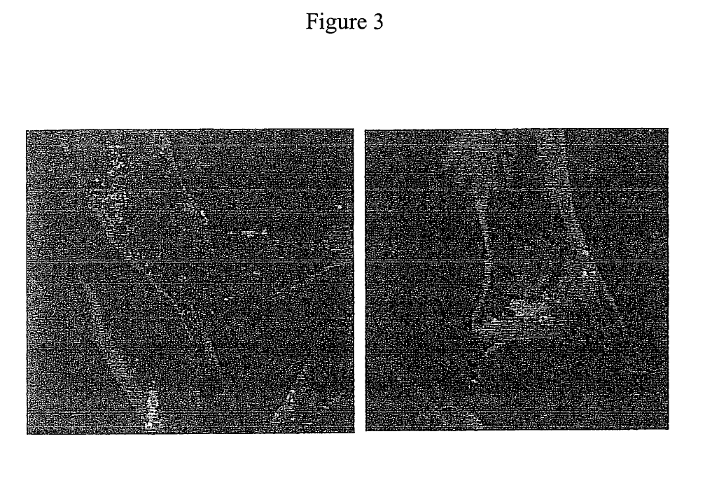

To determine the ability of compounds of the invention to carry siRNA into cells, a 4G-SMOC compound of the invention was complexed with fluorescently labelled cdc7 targeted siRNA. The cellular uptake, followed via live microscopy in a WI-38 human diploid fibroblast cell line is shown as FIG. 3.

### Example 5

### Comparison of Knockdown Efficiency for mRNA

The efficiency of knockdown of cdc7 mRNA using a complex of a SMOC compound of the present invention with siRNA was compared with the efficiency with a lipofectamine complex of the same siRNA.

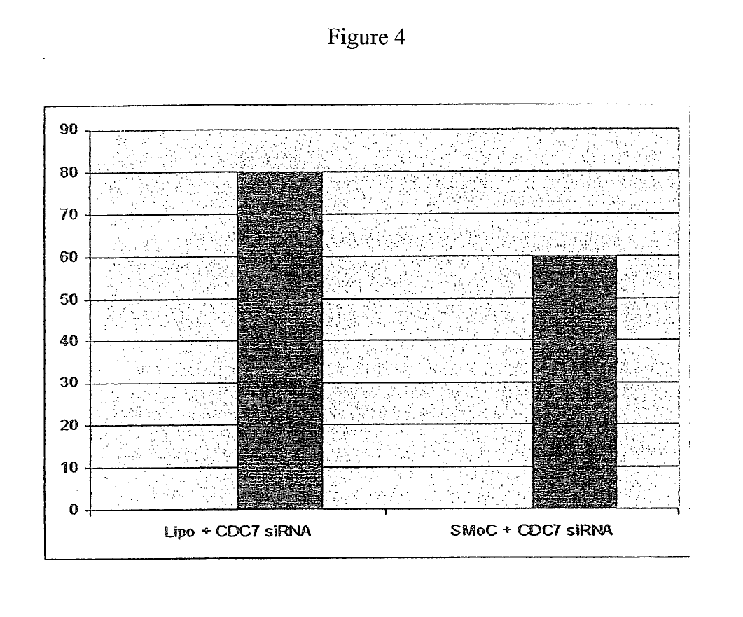

The results of this comparison are shown as FIG. 4.

### Example 6

### Comparative Analysis of Protein Level

The protein level (as assessed by Westerns) using a complex of a SMOC compound of the present invention with siRNA was compared with the protein level (as assessed by Westerns) with a lipofectamine complex of the same siRNA.

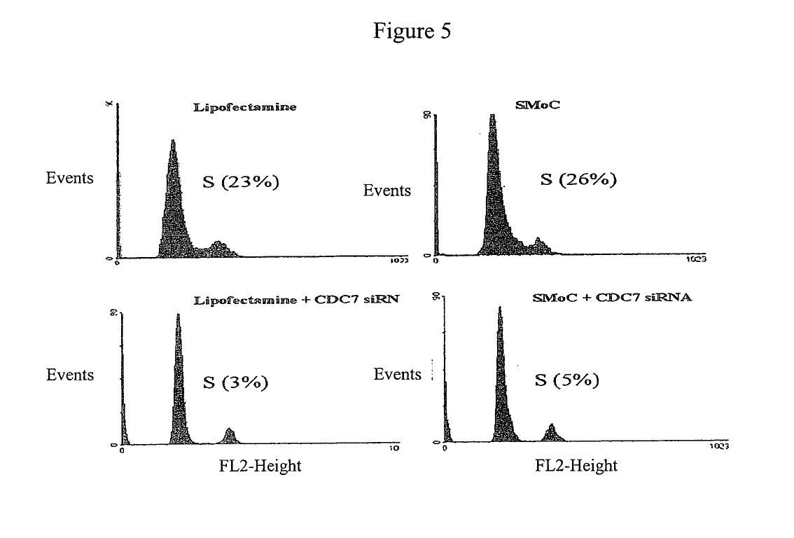

The results of this comparison are shown as FIG. 5.

### Example 7

### Assessment of Progression Through Cell Cycle

The progression through the cell cycle using a complex of a SMOC compound of the present invention with siRNA was compared with the progression through the cell cycle with a lipofectamine complex of the same siRNA.

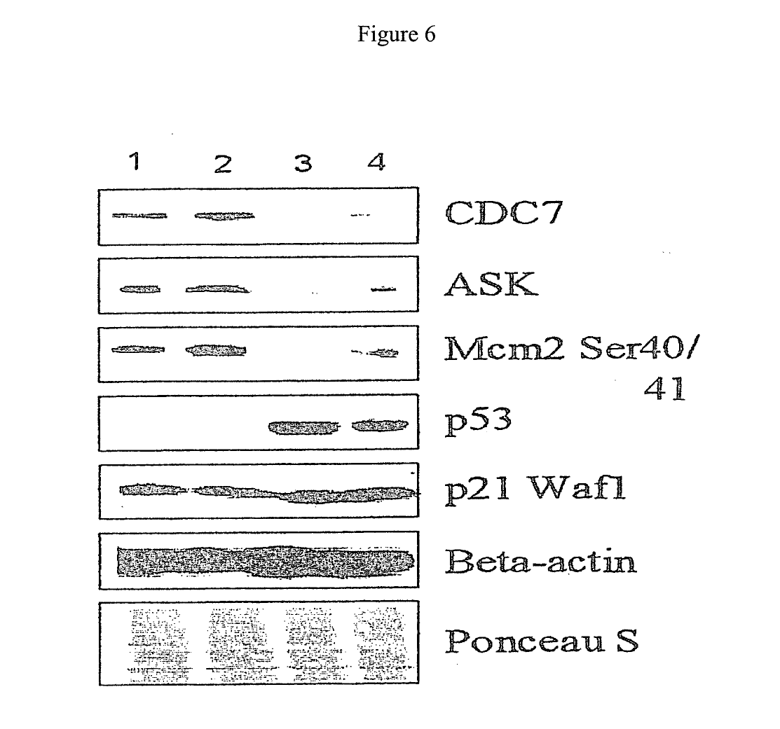

The results of this comparison are shown as FIG. 6.

1. A compound of formula I, or a pharmaceutically acceptable salt thereof, 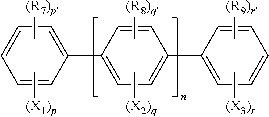
wherein
X₁, X₂ and X₃ are each independently 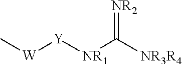
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, CN, NO₂ and COOH;
q is 1, 2, 3 or 4;
q′ is 0, 1, 2 or 3, where q+q′ equals 4;
p and r are each independently 1, 2, 3, 4 or 5;
p′ and r′ are each independently 0, 1, 2, 3 or 4, where p+p′ and r+r′ each equal 5; and
n is 0, 1, 2, 3, 4, 5 or 6.

2. A compound according to claim 1 wherein Y is a C₁₋₁₀ alkylene group, a C₂₋₁₀ alkenylene group or a C₂₋₁₀ alkynylene group.

3. A compound according to claim 1 wherein W is O.

4. A compound according to claim 2 wherein Y is CH₂CH₂.

5. A compound according to claim 1 wherein R₁, R₂, R₃ and R₄ are each independently selected from H, or a butyloxycarbonyl (Boc) protecting group.

6. A compound according to claim 1 wherein p, q and r are each independently 1 or 2.

7. A compound according to claim 1 wherein X₁, X₂ and X₃ are the same and are all 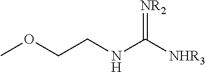
and where R₂ and R₃ are each independently H or a Boc protecting group.

8. A compound according to claim 1 wherein R₇, R₈ and R₉ are all H.

9. A compound according to claim 8 wherein said compound is of formula Ia, Ib, Ic, Id, or Ie 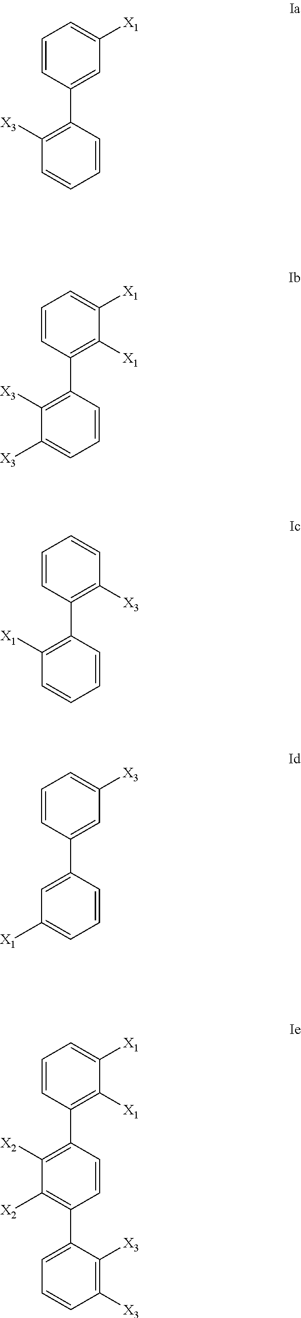
wherein X₁, X₂ and X₃ are as defined in claim 1.

10. A compound according to claim 9 wherein X₁, X₂ and X₃ are the same.

11. A compound according to claim 1 which is selected from the following:

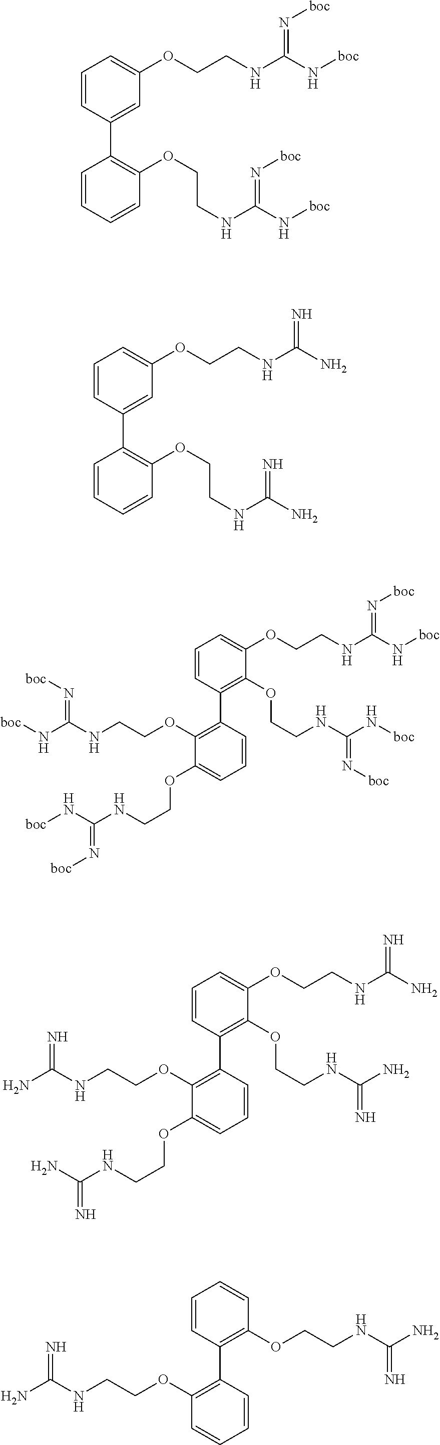

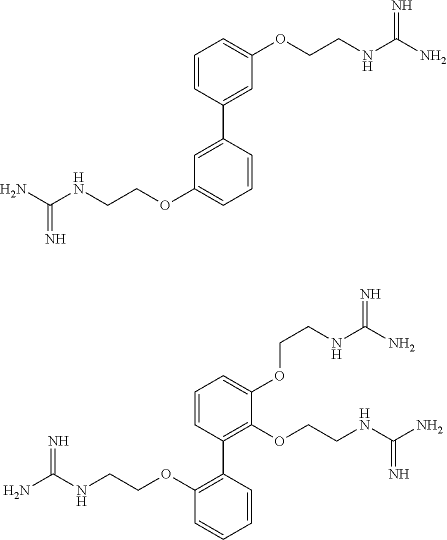

12. A compound according to claim 1, where n is 1.

13. A compound according to claim 12, which is:

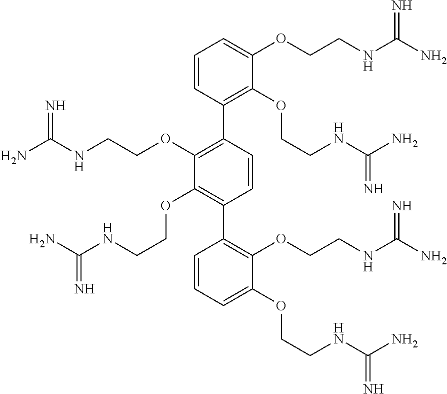

14. A conjugate (U) comprising a compound of formula I as defined in claim 1 and a cargo moiety selected from a protein, a peptide, an oligonucleotide, a nucleotide, a diagnostic agent and a drug.

15. The conjugate according to claim 14, wherein the cargo moiety is RNA.

16. The conjugate according to claim 15, wherein the RNA is siRNA.

17. A compound of formula A₁-B-A₂, or a pharmaceutically acceptable salt thereof, wherein A₁ and A₂ may be the same or different and are 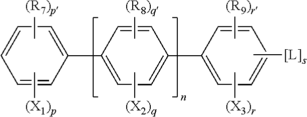
wherein:
R₇, R₈, R₉, X₁, X₂, X₃, n, p, p′, q, q′, r and r′ are as defined in claim 1;
s is 0 or 1;
r+r′+s is 5;
L is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore, 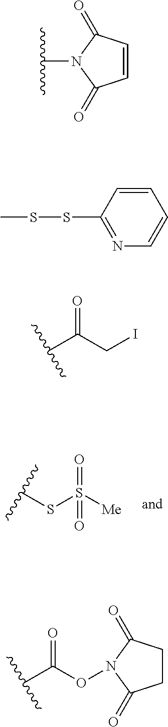
or R₅, R₆ and the nitrogen to which they are attached together form; and 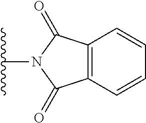
B is —Y′-D-Y′-E-Y′-F-Y′—, wherein:
each Y′ is the same or different and represents a direct bond or a C₁-C₄ alkylene group, which is unsubstituted or substituted with one or more substituents selected from halogen atoms, —NH₂ and —OH groups;
D and F are each independently chosen from:
(i) —NR₁₁C(O)—, —C(O)NR₁₁—, —C(O)O—, —OC(O)—, —O—, —S—, —CO—, —S(O), —S(O)₂—, wherein R₁₁ and R₁₂ each independently represents a hydrogen atom or a straight or branched C₁₋₆ alkyl group, which is unsubstituted or substituted with one or more substituents selected from halogen atoms, —NH₂ and —OH groups; or
(ii) 5- to 6-membered heteroaryl or 5- to 6-membered non-aromatic heterocyclic groups, which groups are optionally substituted by one, two or three substituents selected from halogen atoms, hydroxy, —SH, —NH₂, nitro, cyano, straight or branched C₁₋₆ alkyl, straight or branched C₁₋₆ alkoxy, which C₁₋₆ alkyl and C₁₋₆ alkoxy groups may themselves be substituted with one or more substituents selected from halogen atoms, —NH₂ and —OH groups; and
E is chosen from —S—S— and a direct bond.

18. A compound according to claim 17, wherein s is 0.

19. A compound according to claim 17, wherein A₁ and A₂ are the same.

20. A compound according to claim 17, wherein the compound of formula A₁-B-A₂ is of formula XIa and s is 0, represented by the formula: 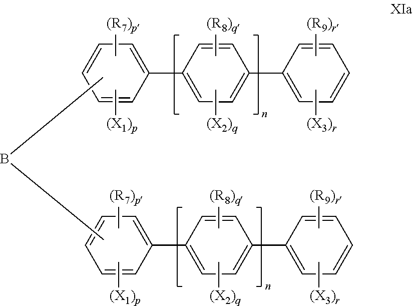
wherein B, R₇, R₈, R₉, X₁, X₂, X₃, n, L and s are as defined in claim 17;
p is 1, 2, 3, or 4
p′ is 0, 1, 2, or 3 where p+p′ equals 4;
q is 1, 2, 3 or 4;
q′ is 0, 1, 2 or 3, where q+q′ equals 4;
r is 1, 2, 3, 4, or 5;
r′ is 0, 1, 2, 3, or 4 where r+r′ equals 5.

21. A compound according to claim 17, wherein B is —Y′—NHC(O)—Y′—S—S—Y′—C(O)NH—Y′—, —Y′—NHC(O)—Y′—Y′—C(O)NH—Y′—or

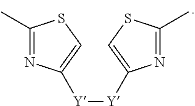

22. A compound according to claim 17, which is selected from

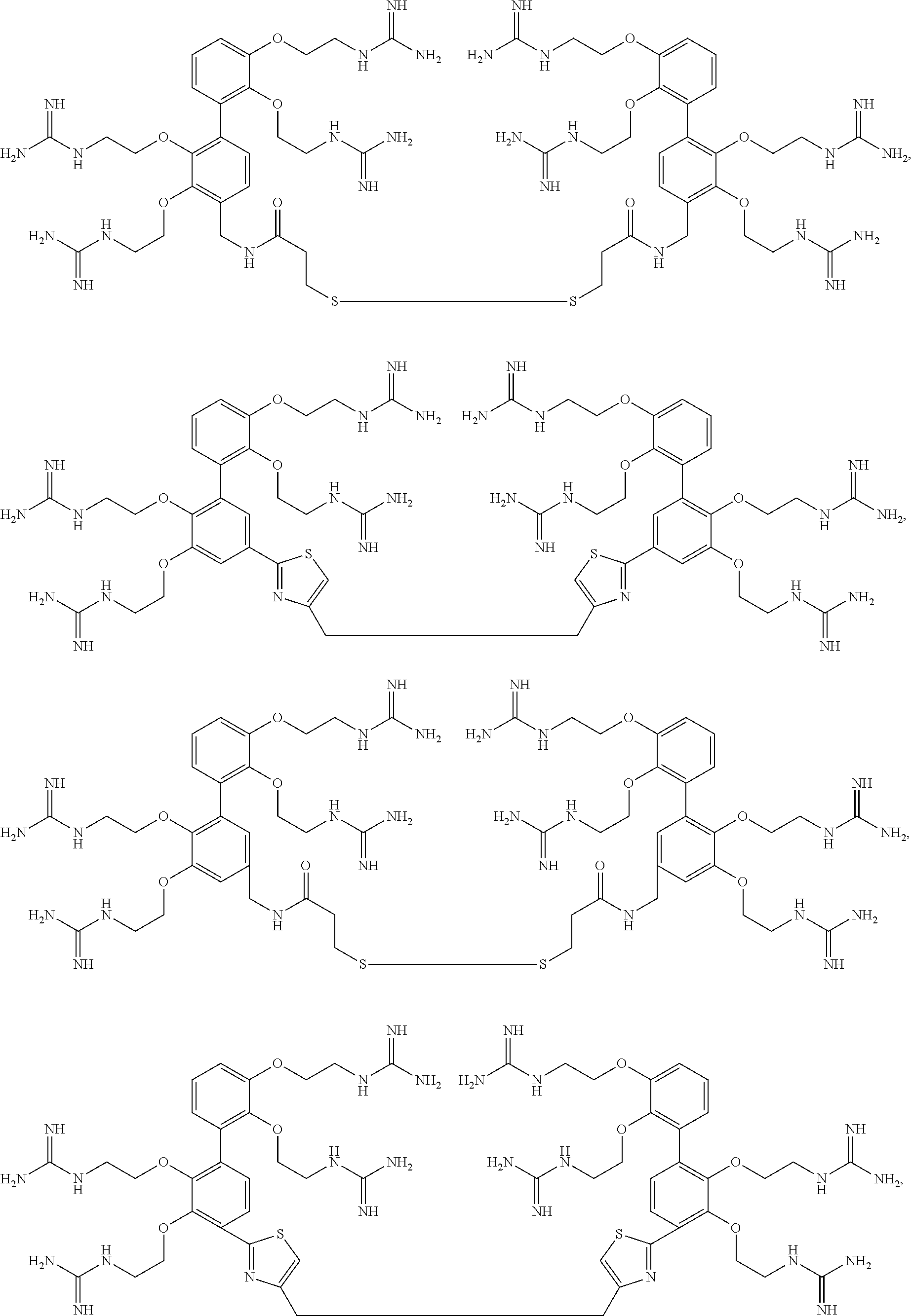

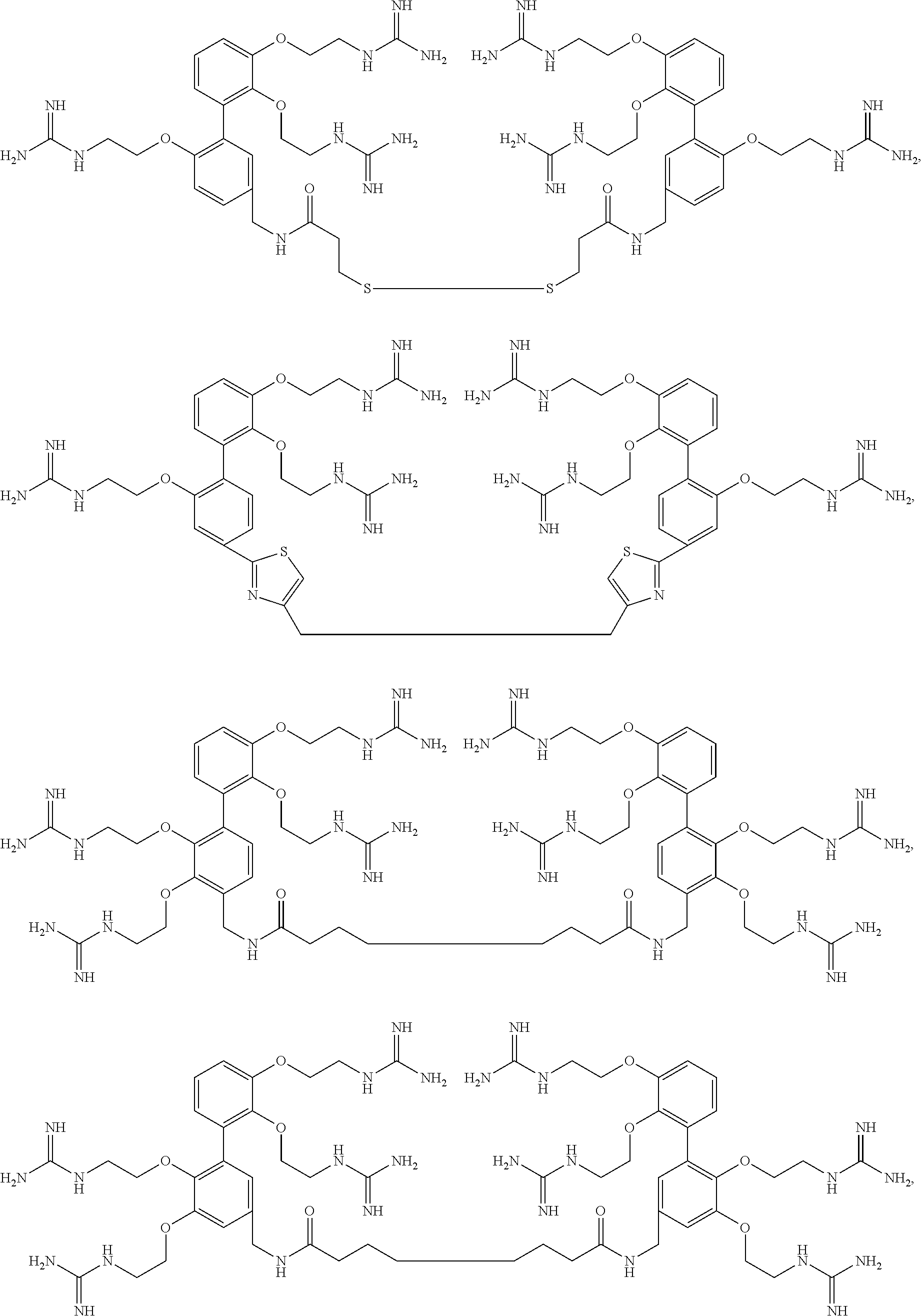

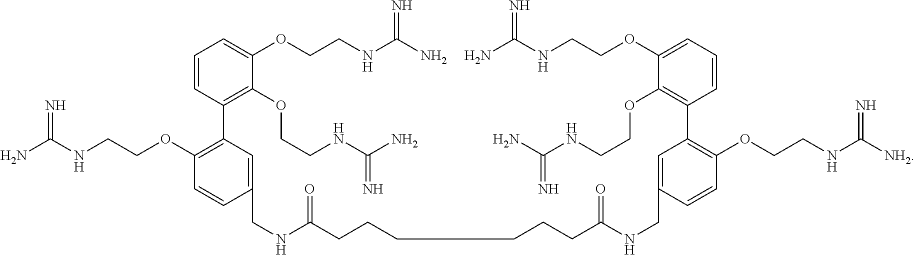

23. A conjugate (V) comprising a compound of formula A₁-B-A₂ as defined in claim 17 and a cargo moiety selected from a protein, a peptide, an oligonucleotide, a nucleotide, a diagnostic agent and a drug.

24. A process for preparing a conjugate (W), which process comprises reacting a compound of formula A₁-B-A₂ as defined in claim 17 or a pharmaceutically acceptable salt thereof, in which s is 1 and L is other than a moiety —(Z)ₘ-phthalimide, wherein Z and m are as defined in claim 17, with a cargo moiety selected from a protein, a peptide, an oligonucleotide, a nucleotide, a diagnostic agent and a drug.

25. A conjugate (W) obtainable by reacting a compound of formula A₁-B-A₂ as defined in claim 17 or a pharmaceutically acceptable salt thereof, in which s is 1 and L is other than a moiety —(Z)ₘ-phthalimide, wherein Z and m are as defined in claim 17, with a cargo moiety selected from a protein, a peptide, an oligonucleotide, a nucleotide, a diagnostic agent and a drug.

26. A pharmaceutical composition comprising a compound according to claim 1 and a pharmaceutically acceptable excipient, diluent or carrier.

27. (canceled)

28. A method for introducing a cargo moiety into a cell, said method comprising contacting said cell with a conjugate according to claim 14.

29. A pharmaceutical composition comprising a conjugate according to claim 14 and a pharmaceutically acceptable excipient, diluent or carrier.


SEQUENCE LISTING (2 sequences)

SEQ ID NO: 1 - protein, 4 aa, Homo sapiens
RRRR

SEQ ID NO: 2 - protein, 8 aa, Homo sapiens
RRRRRRRR